(19) United States
(12) Patent Application Publication
(10) Pub. No.: US 2026/0157735 A1
(43) Pub. Date: Jun. 11, 2026

(54) SAMPLE COLLECTION DEVICE

(71) Applicant: Exact Sciences Corporation, Madison, WI (US)

(72) Inventors: Kevin T. Conroy, Madison, WI (US); Michael J. Domanico, Middleton, WI (US); Keith D. Fourrier, Madison, WI (US); Bridget Z. Gagrat, Madison, WI (US); Timothy E. Higgins, Verona, WI (US)

(21) Appl. No.: 19/123,746

(22) PCT Filed: Oct. 26, 2023
(86) PCT No.: PCT/US2023/077955

§ 371 Date: Apr. 23, 2025

Related U.S. Application Data
(60) Provisional application No. 63/381,031, filed on Oct. 26, 2022.

(30) Foreign Application Priority Data
Apr. 26, 2023 (WO) WIPO132201

(51) Int. Cl. A61B 10/00

(52) U.S. Cl. CPC A61B 10/0038; A61B 10/0096

(57) ABSTRACT

A device for collecting a metered sample from specimen material, particularly soft or semisolid materials such as stool, foods, soil, environmental, and/or industrial materials, and methods and kits for using the technology for collection of metered samples of material for analysis.

The present application claims priority to U.S. Provisional Application Ser. No. 63/381,031, filed Oct. 26, 2022, and Industrial Design Application Ser. No.______, filed Apr. 26, 2023, now International Registration No. DM/231747, each of which is incorporated herein by reference in its entirety.

### FIELD OF THE INVENTION

Provided herein is technology relating to collecting and preparing samples. For example, the technology relates particularly, but not exclusively, to devices, systems, and kits that allow for the collection and preparation of a sample of soft matter compositions, e.g., fecal material, for analysis, and methods using the devices.

### BACKGROUND OF THE INVENTION

Producing a defined volume or mass of a sample for subsequent testing is an important initial step in many settings, e.g., in the food industry, for environmental testing, in medicine, etc. For instance, in a medical setting, laboratory examination of soft matter compositions, e.g., stool samples, is an important component of some diagnoses. In the food testing settings, sampling soft matter compositions may comprise sampling prepared foods, e.g., canned entrees, spreadable or spoonable foods such as nut butters, cheeses, yogurts, pastes, etc., and in environmental testing settings, soft matter compositions to be sampled may comprise wet soils, clays, sludges, etc.

Diagnostic testing often requires isolating and assaying biomolecules from a specimen (tissue, body fluid, stool, e.g.) collected from a subject. Biomolecules for analysis include, e.g., nucleic acids, proteins, fats, or other molecules present in the specimen.

Stool samples for testing are preferably collected by a subject in the home, at the convenience of the subject. For reliability and compliance, devices used for sample collection at home by an untrained person (i.e., a person not trained in medical sample collection) are preferably easy to use correctly, and can be reliably sealed for shipping the sample to a medical facility. For samples that some subjects may find distasteful to handle, e.g., stool samples, devices are preferably configured to minimize exposure of the subject to the sample material. Sample integrity and reproducibility of the sample size are also critically important for the subsequent processing and testing steps.

Available devices for at-home stool sample collection are typically either larger or smaller than necessary for collecting amounts of stool suitable for nucleic acid analysis, especially for analysis of human nucleic acids in the sample (e.g., free nucleic acids, nucleic acids in colorectal cells from the human subject who produced the stool sample). Large kits, typically configured to collect whole stools from a subject for nucleic acid testing, are more costly to manufacture and to ship. Further, the collected samples and the devices used are all discarded after testing, and all must be handled and disposed of as hazardous waste. Thus, collection of larger samples produces a larger outflow of hazardous waste. Further, only a portion of a whole stool sample, e.g., typically no more than 10 grams, is required to produce sufficient quantities of human nucleic acids or other analytes for analysis, while whole stool specimens collected using large containers may provide hundreds of grams of material.

Additionally, collection of whole stool samples creates a large variation in the ratio between the stool sample and the stabilizing buffers added at home by the subject prior to returning the collected sample. For example, whole stool specimens collected at home vary in mass from 1 to nearly 1000 grams. However, a fixed volume of stabilizing buffer is typically provided to subjects to stabilize the whole stool specimen after collection and during transport. If the specimen is small, the buffer volume may over-dilute the sample, resulting in low yields of nucleic acids or other analytes. If the specimen is large, the amount of stabilizing buffer may be inadequate to properly stabilize the nucleic acids or other analytes in the sample. Still further, processing of whole stool samples in large collection containers, e.g., homogenizing samples in the buffer prior to nucleic acid extraction, requires larger, specialized laboratory equipment.

Available smaller devices are typically configured for immunoassay of blood proteins (fecal immunochemical test, or “FIT”), a procedure requiring a very small amount of stool, e.g., typically a few milligrams of stool that can be gathered from a whole stool with, e.g., a small spoon, paddle, brush, swab or the like. Thus, devices configured to collect a stool specimen for FIT testing typically do not collect enough stool for analysis of the human nucleic acids in the sample.

Some intermediate-sized devices exist, typically configured for analysis of non-human nucleic acids and other materials, e.g., for analysis of bacterial or viral materials, for infectious disease or gut microbiome analysis. These types of devices typically use a spoon-like implement to collect a small portion of stool, typically about 1 gram or less. Amounts of microbial nucleic acids are typically present in stool samples in amounts that vastly exceed the amounts of human nucleic acids in the same fecal sample. Thus, a sample of 1 gram or less is typically suitable for microbial analysis while being insufficient for analysis of nucleic acids from the human producing the stool specimen.

### SUMMARY OF THE INVENTION

Provided herein are technologies for collecting samples of soft matter compositions wherein the samples have a defined size or are of a size within an acceptable size range for analysis of an analyte in the sample. In particular, the technology provides devices, kits and methods relating to collection of metered samples from soft matter compositions, e.g., stool specimens. Embodiments of the technology include but are not limited to:

1. A device for collecting and containing a metered sample, the device comprising a sampler wand comprising a sampler head, wherein the sampler head comprises:
i) a sampler head side wall comprising a plurality of ports therethrough;
ii) a sampler head closed end; and
iii) a sampler head opening bounded by a distal edge;
wherein the sampler head side wall, sampler head closed end and sampler head opening define and contain a sampler head interior void having an interior volume.

2. The device of embodiment 1, wherein the sampler head opening is on a plane defined by the distal edge.

3. The device of embodiment 1, wherein the sampler head has a sampler head central axis, wherein the sampler head opening has a center point on the sampler head central axis.

4. The device of any one of embodiments 1-3, wherein the sampler wand comprises a cap.

5. The device of embodiment 4, wherein the cap is attached to the sampler head, preferably to the sampler head closed end, by a stem.

6. The device of embodiment 4 or embodiment 5, wherein the cap has a center point on a cap central axis, and wherein the sampler wand has a wand central axis defined by the center point of the cap and the center point of the sampler head opening.

7. The device of any one of embodiments 1-6, wherein the distal edge of the sampler head comprises one or more teeth.

8. The device of any one of embodiments 1-7, wherein the sampler head interior void is sized to contain from about 0.1 grams to about 50 grams of sample material, preferably stool sample material.

9. The device of embodiment 8, wherein the sampler head interior void is sized to contain about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams, including any intermediate fractions thereof, of sample material, preferably stool sample material.

10. The device of embodiment 9, wherein the sampler head interior void is sized to contain about 1, 2, 3, 4, 5, 6, 7, 8, 9, 10, 11, 12, 13, 14, or 15 grams, including any intermediate fractions thereof, of sample material, preferably stool sample material.

11. The device of any one of embodiments 4-10, further comprising a collection tube comprising a bottom wall joined to a tube wall, wherein a top edge of the collection tube defines a tube opening.

12. The device of embodiment 11, wherein the cap comprises a cap engagement portion and the collection tube comprises a mated engagement portion.

13. The device of embodiment 12, wherein the cap engagement portion comprises cap threads and wherein the mated engagement portion comprises tube threads.

14. The device of embodiment 12 or embodiment 13, wherein when the cap engagement portion and the mated engagement portion are engaged, the sampler wand and the collection tube form a sealed device.

15. The device of embodiment 14, wherein the collection tube has an interior void bounded by the tube wall, the bottom wall, and a plane defined by the top edge, the tube interior void having an interior void maximum volume of at least 5 mL, preferably between about 5 mL and 250 mL, including any intermediate number of mL or fractions thereof, preferably between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof.

16. The device of embodiment 14 or embodiment 15, wherein the bottom wall of the collection tube comprises a displacing member projecting into the interior void of the collection tube.

17. The device of embodiment 16, wherein the collection tube has a tube central axis defined by center points of the collection tube opening and of the displacing member, and wherein the cap has a center point on a cap central axis, and wherein the sampler wand has a wand central axis defined by the center point of the cap and the center point of the sampler head opening.

18. The device of embodiment 17, wherein the cap has a center point on a cap central axis, and wherein the sampler wand has a wand central axis defined by the center point of the cap and the center point of the sampler head opening, and wherein, when the sampler wand and the collection tube form a sealed device, the wand central axis and the tube central axis are collinear.

19. The device of any one of embodiments 16-18, wherein, when the sampler wand and the collection tube form a sealed device, the displacing member of the collection tube is positioned within the sampler head and fills at least a portion of the sampler head interior void.

20. The device of embodiment 19, wherein, when the sampler wand and the collection tube form a sealed device, the displacing member of the collection tube fills at least 50%, 55%, 60%, 65%, 70%, 75%, 80%, 85%, 90% 95%, 96%, 97%, 98%, 99% of the interior volume of the sampler head interior void.

21. The device of embodiment 20, wherein the displacing member of the collection tube fills substantially all of the sampler head interior void.

22. The device of any one of embodiments 16-21, wherein the device has a primary remaining volume of the interior void of the collection tube that equals the interior void maximum volume less the interior void volume displaced by a sampler head and sampler stem.

23. The device of embodiment 22, wherein a ratio (v:v) of the interior void of the sampler head to the primary remaining volume of the interior void of the collection tube is at least about 1:2, preferably between about 1:2 and 1:300, preferably between about 1:2 and 1:100, preferably between about 1:2 and 1:75, 1:50, 1:40, 1:30, 1:20, 1:10, or 1:5.

24. The device of any one of embodiments 16-23, wherein the device further comprises a volume of solution contained in the collection tube.

25. The device of embodiment 24, wherein a ratio (v:v) of the volume of the interior void of the sampler head to the volume of solution in the collection tube is at least about 1:2, preferably between 1:2 and 1:300, preferably between 1:2 and 1:100, preferably between 1:2 and 1:75, 1:50, 1:40, 1:30, 1:20, 1:10, or 1:5.

26. The device of embodiment 24 or embodiment 25, wherein the solution comprises one or more agents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

27. The device of any one of embodiments 16-23, wherein the device further comprises at least one dried agent contained in the collection tube.

28. The device of embodiment 27, wherein the at least one dried agent comprises one or more reagents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt;
XV) an inhibitor binding agent; and
xvi) a desiccant.

29. The device of any one of embodiments 22-28, wherein the device has a secondary remaining volume of the interior void of the collection tube that equals the maximum volume of the interior void of the collection tube less the volume displaced by a sampler head and sampler stem, less the volume of the sampler head interior void.

30. The device of embodiment 29, wherein a ratio (v:v) of the interior void of the sampler head to the secondary remaining volume of the interior void of the collection tube is at least about 1:2, preferably between about 1:2 and 1:300, preferably between about 1:2 and 1:100, preferably between about 1:2 and 1:75, 1:50, 1:40, 1:30, 1:20, 1:10, or 1:5.

31. A method of collecting a metered sample of material, comprising:
a) providing a device comprises a sampler wand comprising a sampler head, wherein the sampler head comprises:
   i) a sampler head side wall comprising a plurality of ports therethrough;
   ii) a sampler head closed end; and
   iii) a sampler head opening bounded by a distal edge;
wherein the sampler head side wall, sampler head closed end and sampler head opening define and contain a sampler head interior void having an interior volume; and
b) collecting an amount of the material within the interior void of the sampler head.

32. The method of embodiment 31, wherein the sampler head opening is on a plane defined by the distal edge.

33. The method of embodiment 31 or embodiment 32, wherein the sampler wand comprises a cap.

34. The method of embodiment 33, wherein the cap is attached to the sampler head, preferably to the sampler head closed end, by a stem.

35. The method of embodiment 33 or embodiment 34, wherein the cap has a center point on a cap central axis, and wherein the sampler wand has a wand central axis defined by the center point of the cap and the center point of the sampler head opening.

36. The method of any one of embodiments 31-35, wherein the distal edge of the sampler head comprises one or more teeth.

37. The method of any one of embodiments 31-36, wherein the sampler head interior void is sized to contain from about 0.1 grams to about 50 grams of sample material, preferably stool sample material.

38. The method of embodiment 37, wherein the sampler head interior void is sized to contain about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams, including any intermediate fractions thereof, of sample material, preferably stool sample material.

39. The method of embodiment 37, wherein the sampler head interior void is sized to contain about 1, 2, 3, 4, 5, 6, 7, 8, 9, 10, 11, 12, 13, 14, or 15 grams, including any intermediate fractions thereof, of sample material, preferably stool sample material.

40. The method of any one of embodiments 33-39, wherein the device further comprises a collection tube comprising a bottom wall joined to a tube wall, wherein a top edge of the collection tube defines a tube opening.

41. The method of embodiment 40, wherein the cap comprises a cap engagement portion and the collection tube comprises a mated engagement portion.

42. The method of embodiment 41, wherein the cap engagement portion comprises cap threads and wherein the mated engagement portion comprises tube threads.

43. The method of embodiment 41 or embodiment 42, wherein, when the cap engagement portion and the mated engagement portion are engaged, the sampler wand and the collection tube form a sealed device.

44. The method of any one of embodiments 33-43, further comprising:
c) inserting the sampler head into the collection tube.

45. The method of embodiment 44, wherein the collection tube has an interior void bounded by the tube wall, the bottom wall, and a plane defined by the top edge, the tube interior void having a maximum volume of at least 5 mL, preferably between about 5 mL and 250 mL, including any intermediate number of mL or fractions thereof, preferably between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof.

46. The method of any one of embodiments 40-45, wherein the bottom wall of the collection tube comprises a displacing member projecting into an interior void of the collection tube.

47. The method of embodiment 46, wherein the collection tube has a tube central axis defined by center points of the collection tube opening and of the displacing member.

48. The method of embodiment 47, wherein the cap has a center point on a cap central axis, and wherein the sampler wand has a wand central axis defined by the center point of the cap and the center point of the sampler head opening, and wherein, when the sampler wand and the collection tube form a sealed device, the wand central axis and the tube central axis are collinear.

49. The method of any one of embodiments 46-48, wherein, when the sampler wand and the collection tube form a sealed device, the displacing member of the collection tube is positioned within the sampler head and fills at least a portion of the sampler head interior void, whereby at least a portion of the amount of material collected in the sampler head interior void is expelled from the sampler head interior void through a port in the sampler head and into the collection tube, wherein the metered sample of the material is selected from:
the amount of material contained in the sampler head interior void; and
the amount of material expelled from the sampler head interior void.

50. The method of embodiment 49, wherein the device further comprises a volume of solution contained in the collection tube.

51. The method of embodiment 50, wherein a ratio of the volume of the metered sample to the volume of solution in the collection tube (v:v), or a ratio of a mass of metered sample to the volume of solution in the collection tube (w:v) is at least about 1:2, preferably between 1:2 and 1:300, preferably between 1:2 and 1:100, preferably between 1:2 and 1:75, 1:50, 1:40, 1:30, 1:20, 1:10, or 1:5.

52. The method of embodiment 50 or embodiment 51, wherein the solution comprises one or more reagents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

53. A kit for collecting a metered sample of material, comprising:
i) a device of any one of embodiments 1-30; and
ii) an agent selected from:
   a solution, and
   a dried agent.

54. The kit of embodiment 53, wherein the device is a sealed device containing the agent.

55. The kit of embodiment 53 or embodiment 54, wherein the agent is a solution comprising one or more reagents from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

56. The kit of embodiment 53 or embodiment 54, wherein the agent is a dried agent comprising one or more reagents from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt;
XV) an inhibitor binding agent; and
xvi) a desiccant.

57. The kit of any one of embodiments 53-56, further comprising one or more of:
a) packaging for shipping, storage, or mailing the device;
b) printed instructions for collecting a metered sample using the device;
c) a collection system for holding specimen material while collecting a metered sample;
d) a holder for holding an unsealed device during collection of a metered sample from a specimen; and
e) a holder for securing a sealed device in a shipping container.

Embodiments of the technology further comprise:

58. A method of processing a stool sample, the method comprising,
a) obtaining a stool sample by the methods of any one of claims 31-52;
b) testing the stool sample for an amount and/or presence of analytes, wherein the analytes comprise one or more of the following:
   i) a nucleic acid
   ii) a protein
   iii) a lipid
   iv) a carbohydrate, and
   v) a metabolite.

### Definitions

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

As used herein, the term “metered” means having a measured quantity that is reproducible with an acceptable range of variation.

As used herein, the term “analyte” is to be construed broadly as any compound, molecule, element, ion, or other substance of interest to be detected, identified, or characterized.

As used herein, the terms “subject” and “patient” refer to an animal, preferably a human, from which a stool specimen is collected. In some instances, the subject is also a “user” (and thus the user is also the subject or patient).

As used herein, the transitional phrase “consisting essentially of” as used in reference to compositions, steps, or other features is to be read as “consisting of” the specified materials, steps, or features, plus only unavoidable additional elements that do not materially affect the basic and novel characteristic(s) of the materials, methods, steps, etc., e.g., unavoidable contaminants, unavoidable steps.

As used herein, the term “comprising” is used interchangeably with “including,” and “containing,” “having,” and “characterized by,” and is inclusive or open-ended and does not exclude additional, unrecited elements, features, or method steps. The term “comprising” means that the named elements are essential, but other elements may be added and still form a construct within the scope of the claim.

The transitional phrase “consisting essentially of” limits the scope of a claim to the specified materials or steps “and those that do not materially affect the basic and novel characteristic(s)” of the claimed invention.

The transitional phrase “consisting of” excludes any element, step, or ingredient not specified in the claim.

As used herein, the term “sample” and “specimen” are used interchangeably, and in the broadest senses. In one sense, sample is meant to include a specimen or culture obtained from any source, as well as biological and environmental samples. Biological samples may be obtained from animals (including humans) and encompass fluids, solids, tissues, and gases. Biological samples include blood products, such as plasma, serum, stool, urine, and the like. Environmental samples include environmental material such as surface matter, soil, mud, sludge, biofilms, water, crystals, and industrial samples. Such examples are not however to be construed as limiting the sample types applicable to the present invention.

As used herein, the term “soft matter composition” is used without limitation to refer to any material or specimen of soft composition, that is preferably non-liquid, and encompasses, e.g., soft solids and semisolids, such as stool, pastes, gels, greases, butters, foams, sludges, clays, tissues, etc.

As used herein, the term “non-liquid” as used in reference to a consistency of a soft matter composition or material to be sampled, refers to a soft material that does not exhibit the characteristics of a liquid, e.g., the characteristic readiness to flow and conform its shape to the shape of a container, and lacking the ability to maintain a fixed shape in the absence of a container.

As used herein, the term “metered sample” refers to a sample having a measured quantity that is reproducible with an acceptable range of variation, e.g., within a range of variation that is acceptable for a purpose for which the metered sample is produced.

As used herein, the term “integral” as used in describing a part or feature of a component refers to a feature or part that is formed from the same material, as part of the process of forming the component (e.g., by molding, carving, or machining). A feature formed as an integral part of a component is distinguished, for example, from a feature that is added to the component after it is formed, e.g., by use of a fastener or adhesive.

As used herein, the term “port” refers to an opening or hole through an element or feature of the device, preferably a hole through a sampler head side wall (10) whereby material (e.g., air, fluid, sample material) that is within sampler head (8) is exposed to an environment outside the sampler head (8), and whereby such material within sampler head (8) may pass between the sampler head interior void (9) to the environment outside the sampler head (8), and vice versa.

As used herein, the term “engagement feature” refers to a feature of a first element of the device (1) configured to interact with a feature of a second element of the device, e.g., to reversibly or irreversibly attach the first element to the second element. For example, in some embodiments, engagement features of a cap and a collection tube are configured to interact with each other to seal the device (1). In some embodiments, an engagement feature of a sampler head (8) is configured to interact with an engagement feature of a collection tube (3), e.g., on the bottom wall (20) and/or displacing member (21) of the collection tube (3), so that the sampler head (8) may be reversibly or irreversibly attached to collection tube (3), e.g., upon sealing of the device. In some embodiments, one part, e.g., a cap (5) has an engagement feature and another part, e.g., a collection tube (3) has a mated engagement feature selected for effective engagement with the engagement feature of the cap. For example, in some embodiments, cap (5) and collection tube (3) have thread features, such that cap (5) is engaged with collection tube (3) by rotation, i.e., by “screwing” the cap onto the collection tube. Other engagement features include but are not limited to bayonet mount elements, snap closure parts, or parts forming a friction seal.

As used herein, a “sealed device” refers to a device (1) having a cap (5) and collection tube (3) in which engagement features on the cap and collection tube are engaged to an extent necessary that a sealing junction (27) prevents passage of fluid, e.g., from inside sealed device (1) to the outside of device (1).

As used herein, the term “substantially” as used in reference to a quality or quantity of a component or feature refers to a quality or quantity in which some degree of variation is permissible, wherein the variation does not materially affect the basic and novel characteristic(s) of the component or feature, or of the claimed embodiment.

As used herein the term “analyte” refers to any component of a sample subject to analysis. Non-limiting examples of analytes include biomolecules, e.g., nucleic acids, proteins, carbohydrates, lipids, metabolites, organic and inorganic compounds; eukaryotic cells, such as human cells or cell components (e.g., tumor-associated cells, tissue cells, blood cells, etc.); bacterial cells or cell components; plant cells or cell components; fungal cells or cell components; including, for each of the aforementioned cells and cell types: whole cells, nucleic acids, organelles, membranes, proteins, small molecules, metabolites, and any molecule, substance, or material produced in or associated with a cell or cell component as a result of, e.g., recombinant engineering, conjugation, transfection, transformation, and/or infection; viral components (e.g., whole viruses, viral nucleic acids, viral proteins, including capsid proteins and viral proteins produced by infected cells, including any molecule, substance, or material produced in, or associated with, a virus or virus component as a result of, e.g., recombinant engineering, conjugation, transfection, transformation, and/or infection; organic compounds, inorganic compounds, etc.

As used herein, the term “kit” refers to any delivery system for delivering materials. In the context of sample collection systems, such delivery systems include systems that allow for the storage, transport, or delivery of devices or the samples collected therewith (e.g., buffers, stabilizers, preservatives, etc. in the appropriate containers) and/or supporting materials (e.g., written instructions for performing a procedure, etc.) from one location to another. For example, kits include one or more enclosures (e.g., boxes) containing the relevant devices and supporting materials. As used herein, the term “fragmented kit” refers to a delivery system comprising two or more separate containers that each contains a subportion of the total kit components. The containers may be delivered to the intended recipient together or separately. For example, a first container may contain materials for sample collection and a buffer, while a second container contains sampling devices, separate shipping materials, etc. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components in a single container (e.g., in a single box housing each of the desired components). The term “kit” includes both fragmented and combined kits.

The term “system” as used herein refers to a collection of articles for use for a particular purpose. In some embodiments, the articles comprise instructions for use, as information supplied on e.g., an article, on paper, or on recordable media (e.g., CD, flash drive, etc.). In some embodiments, instructions direct a user to an online location, e.g., a website, a remote server of a service provider, etc.

As used herein, the term “central axis,” as used in reference to a device or component of a device, refers to an axis about which the device has at least one element having rotational symmetry. For example, in device (1) depicted in FIGS. 1 and 2, central axis (23) is indicated by a dashed line, and is defined by the line between the point at the center of the bottom of the collection tube (3) and the point at the center of the top of the cap (5), when the cap is engaged with the collection tube. In the depicted embodiment, when the device is not assembled, the central axis of sampler wand (2) is defined by the point at the center of the sampler head opening (14) and the center of the top of cap (5), while the central axis of the collection tube (3) is defined by the line between the point at the center of the tube opening (19) and the point at the center of the bottom wall (20) of collection tube (3) and/or a point at the center of the displacing member (21). Embodiments may comprise an asymmetrical feature, e.g., a sampler wand stem (4) or sampler head (8) comprising one or more ridges, bumps, dimples, grooves or other protruding or indenting elements that differ from each other, e.g., in size, shape, and/or distribution such that they are not symmetrical. In such embodiments, a basic or underlying shape of a device component, e.g., a sampler wand stem (4) or sampler head (8) (or other component of a sampler wand or collection tube), may be considered separately from such features. For example, a basic conical or tubular shape of a stem (4) or a bell shape of a sampler head (8), such as those in the embodiments depicted in FIGS. 1 and 7A, respectively, may define a central axis, even if such stems and/or sampler heads further comprise elements that are not symmetrical, e.g., with respect to the central axis.

### BRIEF DESCRIPTION OF THE DRAWINGS

FIG. 1 is a view of an embodiment of the device provided herein, showing a sampler wand (2) and a collection tube (3), with the sampler wand disengaged from the collection tube.

FIG. 2 is a side view of an embodiment of the device provided herein, showing device 1 in a sealed state in which cap (5) of sampler wand (2) is secured to collection tube (3). The stem (4) and sampler head (8) are visible within collection tube (3).

FIGS. 3A and 3B show side views of a sampler wand (2) and a collection tube (3) of an embodiment of the device provided herein, with dimensions shown in inches (FIG. 3A) or in millimeters (FIG. 3B).

FIG. 4A shows a side view and a sectional view of an embodiment of the device provided herein, in which cap (5) is engaged with collection tube (3) to provide a sealed device. The embodiment shown comprises a gasket (25) that provides a seal between cap (5) and the top edge (18) of the collection tube (3).

FIG. 4B shows an exploded diagram of an embodiment of sampler wand (2), comprising a cover (6), grip ring (5a), stem (4) with attached sampler head (8), and gasket (25).

FIG. 5A shows a top view of an embodiment of the device (1).

FIG. 5B shows a side view of an embodiment of the device (1), showing exemplary dimensions (in inches).

FIG. 5C shows a bottom view of an embodiment of the device (1), showing indent (26) in bottom wall (20) of collection tube (3).

FIG. 5D shows a cross-sectional view of the device of FIG. 5A at position A-A.

FIG. 5E shows a side view of an embodiment of the device (1), showing exemplary dimensions (in millimeters).

FIGS. 6A-6C show examples of internal volumes of an embodiment of the device: A) the maximum volume of the interior void (22) of collection tube (3) when empty; B) the primary remaining volume of the interior void (22) of collection tube (3) upon displacement by the stem (4) with sampler head (8); C) the volume of interior void (9) of sampler head (8).

FIG. 7A shows embodiments of the device having different arrangements, patterns and distances of raised or indented features (16) (e.g., bumps, ridges, grooves, dimples, etc.) on the exteriors of exemplary caps. The top panel shows 4 sealed devices comprising sampler wands and collection tubes and the bottom panel shows 4 exemplary sampler wands alone. Different embodiments of caps and different embodiments of sampler heads are shown.

FIG. 7B illustrates different embodiments of stems (4) shown as dashed lines indicating exemplary silhouettes of 3-dimensional stems of different shapes and dimensions. Embodiments of the technology are not limited to any particular stem shape or size. Stems may be round in cross-section, or the cross-section may have a different shape, e.g., an oval or ellipse shape, a regular or irregular polygon (e.g., square, rectangle, triangle, hexagon, preferably a convex polygon), etc. Different parts of a single stem may have different cross-sectional shapes. Stems may comprise one or more ridges, bumps, dimples, grooves or other protruding or indenting elements, and may comprise holes or ports therethrough, as illustrated schematically by the two smaller ovals in embodiment VI in FIG. 7B.

FIG. 8A shows different embodiments of sampler heads.

FIG. 8B shows an embodiment of a sampler head (8) comprising breaks (32) between teeth (13).

FIGS. 9A and 9B (detail view of 9A) illustrate positioning of a sampling tip (e.g., of a pipetting device) with respect to the displacing member (21) in collection tube (3), e.g., such that the displacing member does not impede access by the sampling tip to the bottom of the collection tube.

FIG. 10 illustrates an embodiment of a sealed device in a vertical orientation (with the cap at the top) and containing different volumes of fluid (40, 45, or 50 mL). Arrows indicate the fluid level covering sampler head (8).

FIG. 11 illustrates an embodiment of a sealed device in a horizontal orientation and containing different volumes of fluid (40, 45, or 50 mL). Arrows indicate the fluid level partially or completely covering sampler head (8)

FIG. 12A shows a detail illustration of the joint between a flange portion (4a) of stem (4) (see FIG. 4B) of sampler wand (2) and the top edge (18) of collection tube (3), as sealed by gasket (25), when cap threads (7) are engaged with tube threads (24).

FIG. 12B shows a detail illustration of the sealed joint (27) between a flange portion (4a) of stem (4) (see FIG. 4B) of sampler wand (2) and the top edge (18) of collection tube (3), as sealed without a gasket.

FIG. 12C shows a detail illustration of a sealed joint (27) between a sealing lip (28) on cap (5) and a sealing surface (29) on an inner side wall (30) of collection tube (3), as sealed without a gasket. In some embodiments, sealing lip (28) is molded as an integral part of cap (5).

### DETAILED DESCRIPTION OF THE INVENTION

Embodiments of the invention are described in this Detailed Description of the Invention, and in the Summary above, which is incorporated here by reference. Although the invention has been described in connection with specific embodiments, it should be understood that the invention as claimed should not be unduly limited to such specific embodiments. Those skilled in the art who review this disclosure will readily appreciate that many modifications are possible (e.g., variations in sizes, dimensions, structures, shapes and proportions of the various elements, values of parameters, mounting arrangements, use of materials, orientations, etc.) without materially departing from the novel teachings and advantages of the subject matter of the technology. Accordingly, all such modifications are intended to be included within the scope of the present invention as defined in the appended claims. In particular, although features of the technology are described herein by reference to stool samples in particular, the technology is not limited to stool samples and may be applied to any soft matter composition from which a metered sample may be taken.

Provided herein are sampling devices that find use in collecting samples, e.g., stool samples, that are of suitably reproducible size, in sample amounts that are suitable for analytes (e.g., of human nucleic acids, proteins, etc.) in the sample. The device finds particular use in collecting metered samples for disease screening, e.g., nucleic acid-based or protein-based screening for digestive system inflammation and/or cancer, e.g., colorectal cancer, in a subject.

In addition to facilitating collection by a subject of a sample having a reproducible volume or mass, the device provided herein actively expels collected sample material into a stabilizing buffer within the device, enhancing the stabilizing of analytes within the sample material (cells, proteins, nucleic acids, e.g.), and/or reducing loss of sample material, e.g., by sticking of collected sample to the device.

Provided herein are embodiments of a collection device (1) adapted to comprise one or more of the following features:
1. Collect metered samples from soft specimens, e.g., stool specimens of varying consistencies;
2. Collect metered samples that include sample material from the outer and inner regions of the stool specimen, and optionally from multiple locations in the specimen, e.g., to maximize chances of collecting analytes associated with a condition such as inflammation, cancers, e.g., analytes such as cells, nucleic acids, proteins, lipids, etc., if present;
3. Collect enough sample material to yield nucleic acid quantities sufficient for nucleic acid analysis (e.g., methylation assays, gene expression assays);
4. Actively disperse a metered sample into stabilizing buffer within the device;
5. To contain buffer prior to collection of the metered sample, such that the user does not need to transfer buffer to the device from a separate container; and
6. Contain sufficient buffer to cover a collected metered sample, whether the device is vertical or on its side.

In contrast to tube-type collection devices configured for FIT testing, the device of the technology can capture sufficient amounts of sample material, in particular stool sample material, for analysis of target analytes such as nucleic acids, e.g., human nucleic acids, (e.g., >1, 2, 3, 4, 5, 6, 7, 8, 9, 10, 11, 12, 13, 14, or 15 grams, or any intermediate fraction thereof). In preferred embodiments, the device of the technology provides sufficient sample for at least two independent measurements of analytes or panels of analytes, e.g., of target nucleic acids or panels of target nucleic acids. In contrast to whole-stool bucket-type at-home collection devices, the technology provides sample collection devices that use substantially less material to manufacture, are substantially less cumbersome to package, ship, and process, and produce a substantially smaller amount of downstream waste, including hazardous medical waste, for disposal.

### Device

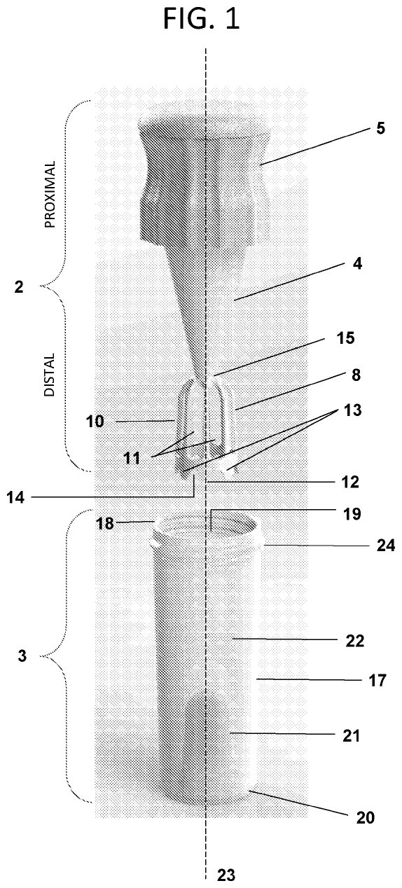

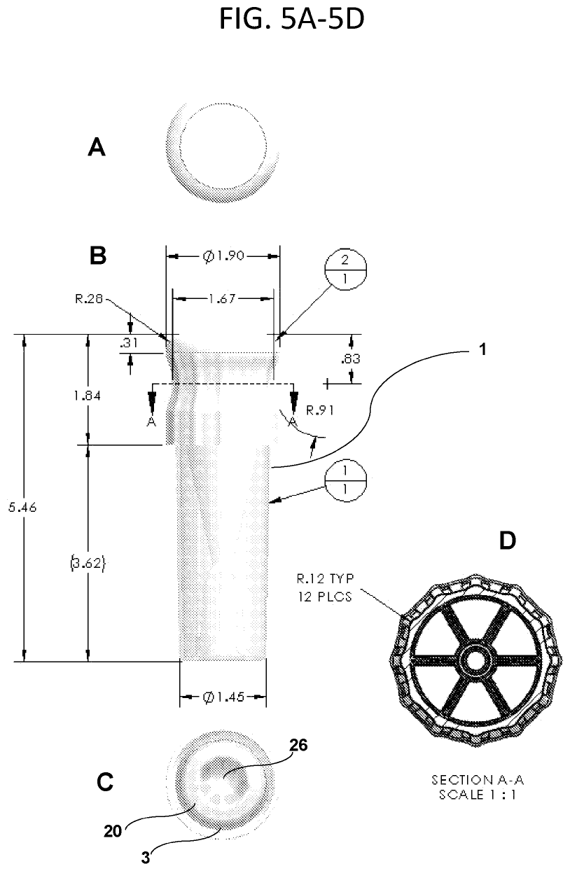

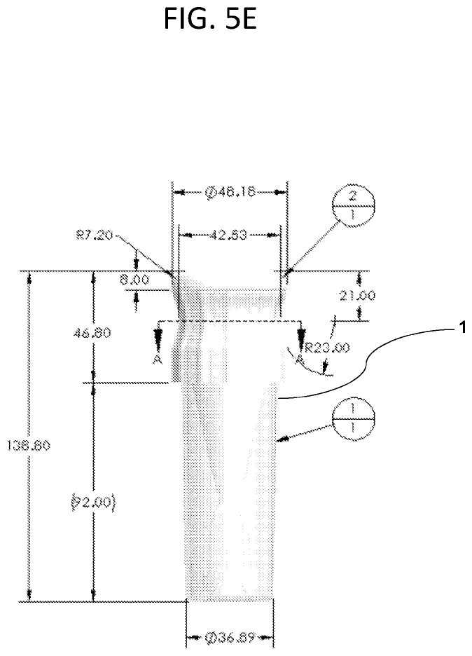

As shown by FIGS. 1-12, the device (1) comprises as principal components a sampler wand (2) and a collection tube (3). Embodiments of the device, particularly in reference to the interactions of these and other components, are described below.

### Sampler Wand

The device comprises as a first component a sampler wand (2). In the embodiment illustrated in FIG. 1, sampler wand (2) comprises a proximal portion adapted for contact with and manipulation by a user and a distal portion that is adapted to capture a metered sample.

In the embodiment illustrated in FIG. 1, sampler wand (2) comprises a stem (4) between the proximal and distal portions of the wand. In the illustrated embodiment, stem (4) connects cap (5) on the proximal portion to sampler head (8) on the distal portion. In some embodiments, sampler wand (2) comprises a cap, while in some embodiments, sampler wand (2) does not comprise a cap.

### Cap

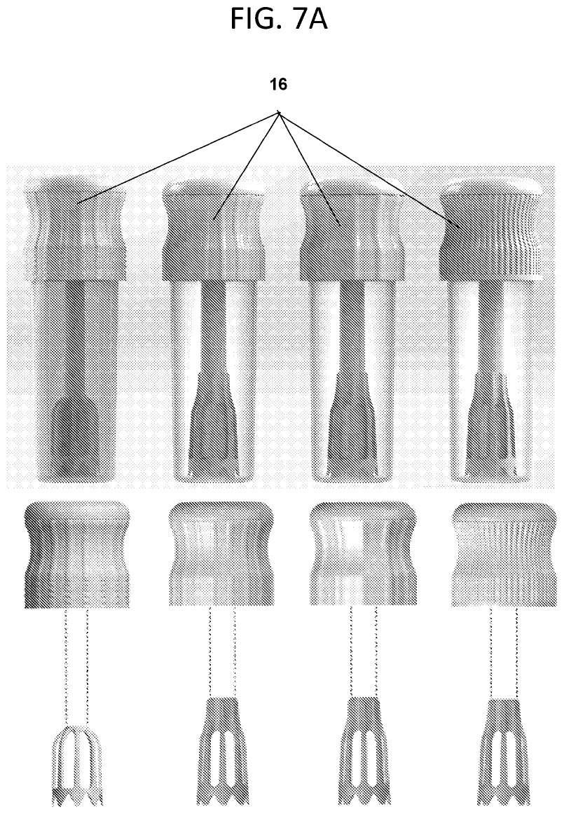

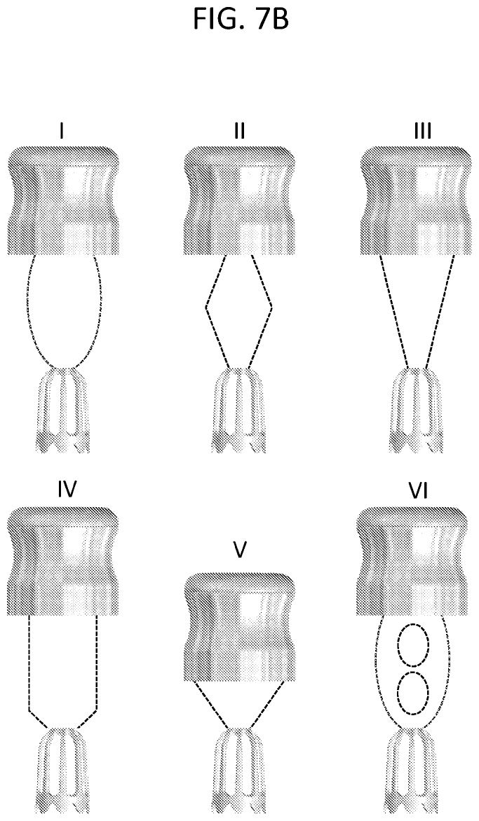

In the embodiment illustrated in FIG. 1, cap (5) is provided. Cap (5) provides a component for gripping or manipulating the sampler wand (2), e.g., by a user or robotic device. Cap (5) further preferably comprises a cap engagement feature, e.g., cap threads (7), configured to engage with a mated engagement feature, e.g., tube threads (24) on a collection tube, wherein when the cap engagement feature is engaged with the mated engagement feature, the cap and tube form a sealed device. In some embodiments, the cap (5) comprises raised or textured features, e.g., bumps and/or ridges as illustrated in FIG. 7A, e.g., useful in gripping and manipulating the sampler wand (2) by the user.

### Sampler Head

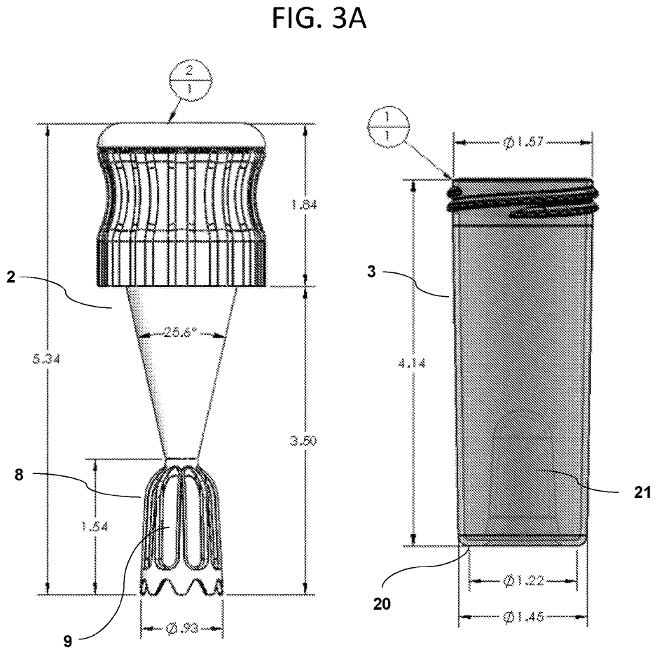

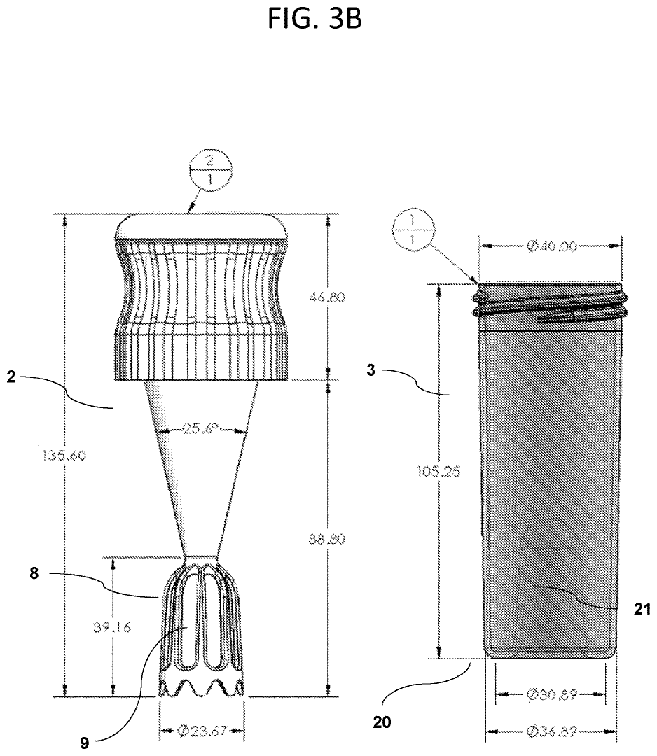

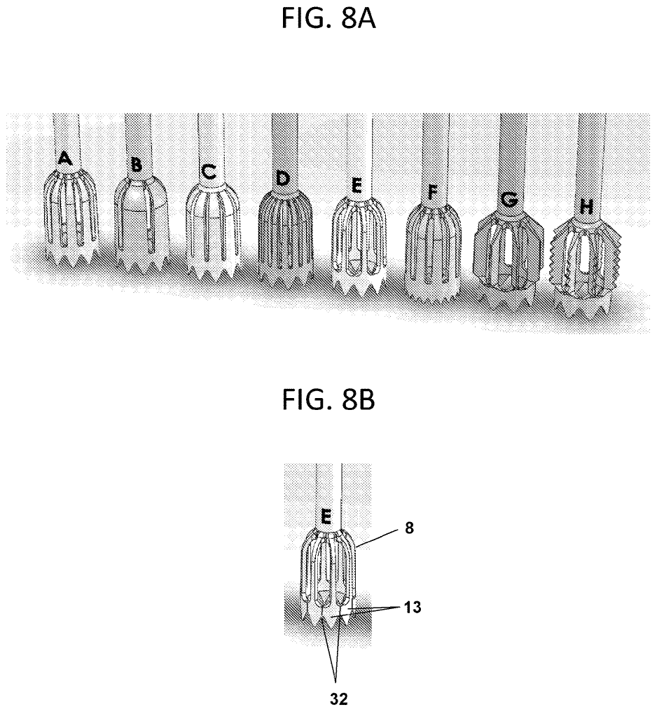

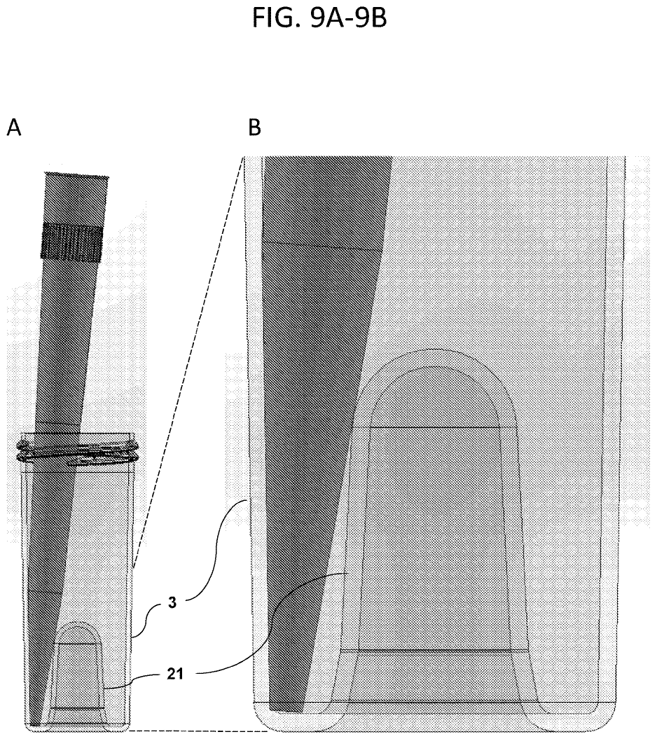

The distal portion of sampler wand (2) comprises a sampler head (8) adapted to capture a metered sample. For example, in the embodiment illustrated in FIG. 1, sampler head (8) comprises a side wall (10) that defines a sampler head opening (14) at the distal end of sampler wand (2). In certain preferred embodiments, side wall (10) comprises a distal edge (12) at the distal end of the sampler wand (2). In some embodiments, the distal edge (12) comprises a plurality of teeth (13). The teeth (13) on distal edge (12) are not limited to any particular shape or edge profile. For example, the teeth (13) may be sharp or pointed, as illustrated in FIG. 8, or the edge profile of the teeth may be squared off, blunted, and/or rounded, e.g., as illustrated in FIGS. 1 and 3, or combinations thereof. The distal edge may comprise 2, 3, 4, 5, 6, 7, 8, 9, 10, 11, 12, 13, 14, 15, 16, 17, 18, 19, 20, or more teeth. In some embodiments teeth (13) are the same length and in some embodiments, some teeth are longer or shorter than neighboring teeth. In some embodiments, teeth (13) on a sampler head (8) are all substantially the same width, while in some embodiments, one or more teeth are wider or narrower than other teeth on the same sampler head (8). Additionally, in some embodiments, a plurality of teeth (13) are evenly distributed such that they are each equidistant from neighboring teeth, while in some embodiments, at least some of the teeth are unevenly arrayed, such that not all teeth are equidistant from neighboring teeth.

In preferred embodiments, the sampler head opening (14) is circumferentially defined by sampler head side wall (10), such that the sampler head opening (14) is circular. In the embodiment illustrated in FIG. 1, the sampler head (8) comprises a closed end (15), at an end of the sampler head attached to stem (4). The sampler head (8) comprises an interior void (9) bounded by side wall (10), closed end (15), and a plane defined by the distal edge (12) of sampler head (8). In embodiments in which the distal edge (12) comprises a plurality of teeth (13), the plane defined by distal edge (12) is defined by points on the ends of 3 or more of teeth, preferably the longest teeth, if the teeth are not all of the same length. In preferred embodiments, the plane of the distal edge (12) is perpendicular to central axis (23). In some embodiments, sampler head side wall (10) defines a sampler head opening (14) that is not circular, but is a different shape, e.g., an oval or ellipse shape, or a regular or irregular polygon (e.g., square, rectangle, triangle, hexagon, preferably a convex polygon), etc.

Sampler head side wall (10) of sampler head (8) comprises at least one port (11), preferably a plurality of ports (11) providing connection between the interior void (9) with the exterior of sampler head (8). In preferred embodiments, sampler head side wall (10) comprises a plurality of ports that are preferably distributed to different positions on the sampler head (8), e.g., the plurality of ports in some embodiments are radially distributed around a central axis of sampler head (8). Additional examples of suitable arrangements of ports (11) on sampler heads are provided in FIG. 8. In other embodiments, sampler head (8) comprises a plurality of ports (11) that are irregularly or asymmetrically arranged on the sampler head (8). The ports (11) are not restricted to any particular shape, and may, for example comprise circular, oval, rectangular, or irregular or other suitable shapes, in any combination or arrangement. As illustrated in FIG. 8B, in some embodiments, the teeth (13) of sampler head (8) are not connected to each other around the circumference of the sampler head side wall (10). For example, in some embodiments, sampler head side wall (10) comprises a break (32) between at least one pair of teeth (13), e.g., connecting a port (11) and distal edge (12) or between a plurality of pairs of teeth (13) or between all teeth (13). In some embodiments, neighboring teeth touch at break (32), while in some embodiments, break (32) provides a gap between neighboring teeth.

### Stem

In preferred embodiments, sampler head (8) is attached to cap (5) by a stem (4). In some embodiments, stem (4) varies in diameter along its length. In some embodiments, stem (4) is tapered along its length. For example, in the embodiment illustrated in FIG. 1, stem (4) has a large diameter at its proximal end and progressively narrows between the proximal portion and the distal portion of the sampler wand (2), presenting as a truncated cone.

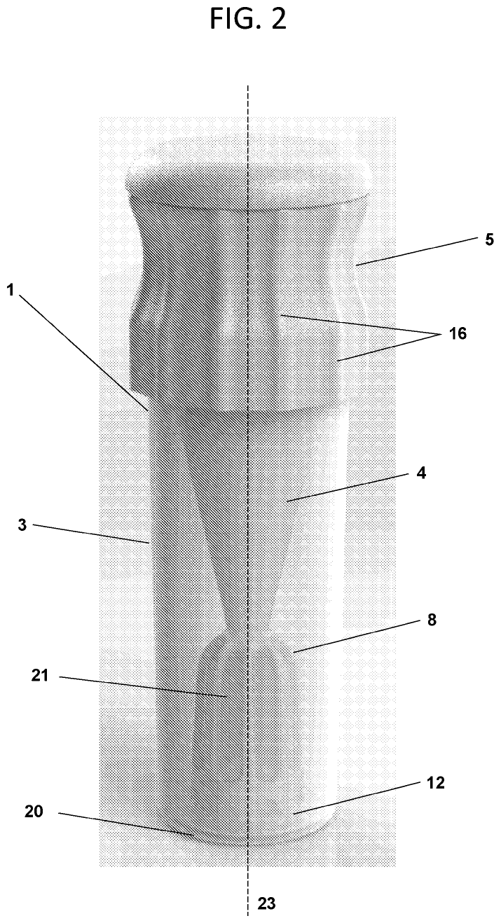

In some embodiments, the shape of stem (4) is selected to provide a particular ratio between the collection tube (3) interior volume and the volume of the sampler head interior void (9), or a particular ratio between a solution and a metered sample when device (1) is in use. In some embodiments, e.g., the shape of stem (4) is selected to maximize the amount of the collection tube interior volume it displaces in a sealed device (1), such that e.g., less solution is required to fill the remaining interior volume of collection tube (3) and/or cover sampler head (8). For example, tapered stem (4) illustrated in FIGS. 1 and 2 displaces more of the interior volume of the collection tube (3) than a slim cylindrical stem, e.g., as shown in the embodiments illustrated in FIG. 7A. Stem (4) may be larger than these exemplary embodiments. For example, stem (4) may be further thickened between cap (5) and sampler head (8), e.g., may have convexly curved or bulging sides.

Stem (4) of the device is not limited to any particular shape. As illustrated in FIG. 7B showing exemplary silhouettes of embodiments of stem (4), any number of shapes may be used in embodiments of the device, so long as the stem positions sampler head (8) at a desired position withing collection tube (3).

As discussed in more detail below, in certain embodiments, use of the device (1) comprises expelling the metered sample from the interior void (9) of the sampler head (8) through the ports (11) during closure of the device, e.g., when cap (5) is screwed on or otherwise sealed to collection tube (3). Thus, in some embodiments, the shape(s) of and/or distribution of ports (11) on sampler head (8) are generally selected to suit a range of expected textural or plastic properties (e.g., firmness, hardness, stickiness, etc.) of a material to be sampled using the device. For example, it may be preferable to use larger ports if a type of metered sample is very firm, as pressing such a sample through very small ports may require an unacceptable amount of pressure. Alternatively, if the material is very soft, it may be preferable to use smaller ports, e.g., to improve containment of the metered sample, and/or to reduce or prevent leakage or loss of the metered sample through the ports before the sampler wand (2) can be inserted into the collection tube (3).

In some embodiments, e.g., for preparing a metered sample from a stool sample, the interior void (9) of a sampler head (8) is sized to collect a suitable amount of sample based on the needs of the test(s) to be conducted, preferably to conduct the needed tests at least twice. As a non-limiting example, for human DNA testing, a metered sample of between about 1 gram and 15 grams finds use. For testing stool for more or different analytes, or for testing other materials (e.g., foods, environmental samples, industrial samples, e.g.) it may be preferable to collect either smaller or larger amounts of a specimen. The volume of the interior void (9) of a sampler head (8) of the instant technology is not limited to any particular size, and may be sized to contain 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more grams of sample, including any intermediate fraction thereof, e.g., 0.1 grams, 0.01 grams, 0.001 grams or multiples thereof.

In preferred embodiments, the metered sample is expelled from the sampler head (8) into a volume of solution in collection tube (3), preferably a stabilizing solution. The volume of solution used for a volume metered sample is preferably selected to be large enough to sufficiently stabilize the metered sample, but to be small enough to avoid excessively diluting the metered sample. In some embodiments, the volume of solution in the collection tube (3) is at least 5 mL, preferably between about 5 mL and 300 mL, including any intermediate number of mL or fractions thereof, preferably between about 5 mL and 300 mL, preferably between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof.

In preferred embodiments, the ratio of the volume of the metered sample to the volume of solution (v:v) e.g., in a collection tube, or the ratio of the mass of the metered sample to the volume of solution (w:v) is at least about 1:2, preferably between 1:2 and 1:300, preferably between 1:2 and 1:100, preferably between 1:2 and 1:75, 1:50, 1:40, 1:30, 1:20, 1:10, or 1:5. In embodiments of the technology, the volume of solution in the collection tube is provided in pre-mixed form, as a solution, while in some embodiments, a volume of solution is provided as a dried agent to be dissolved or resuspended, e.g., by a user of the device, for example, by the addition of a measured amount of water,

In some embodiments, the cap (5) and sampler head (8) are essentially circular in cross section. In preferred embodiments, the stem (4) is also circular in cross section for at least a portion of its length, and the centers of the circular cross-sections of the cap (5), stem (4), and sampler head (8) are aligned on the central axis (23) of the device (1), as illustrated in FIG. 1. In some embodiments, the stem (4) and/or sampler head (8) have a central axis that is not the same as the central axis (23) of the device (1) as a whole.

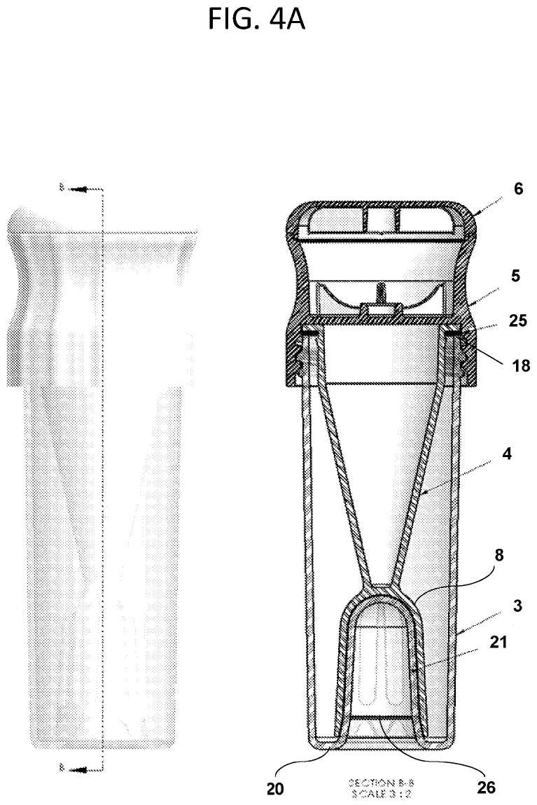

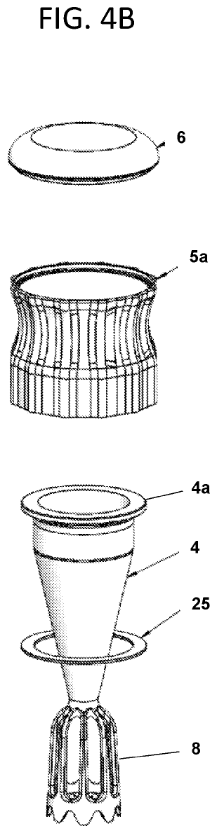

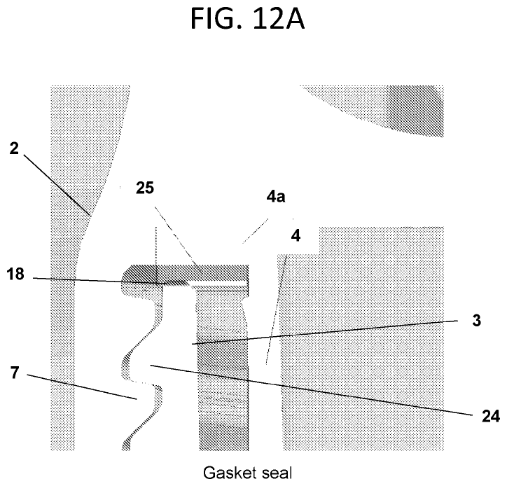

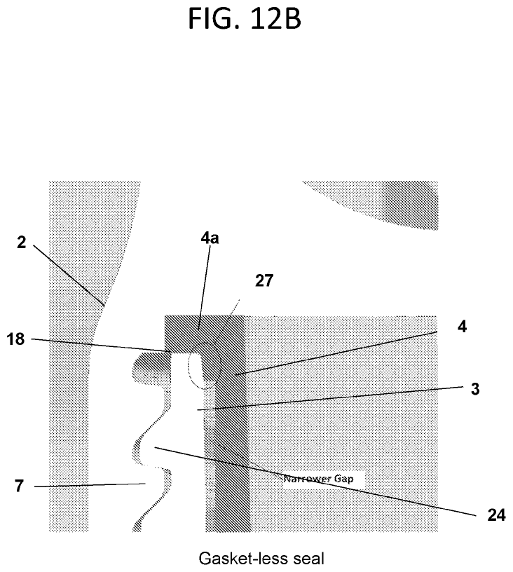

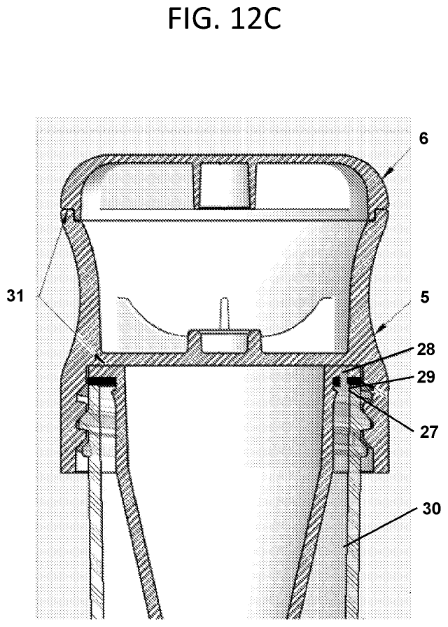

In preferred embodiments, cap (5) is adapted to mate with and seal collection tube (3) (discussed below), at a top edge (18) of the collection tube (3). In preferred embodiments, mated threads on the cap (5) and collection tube (3) engage with each other, e.g. by rotating the cap with respect to the collection tube, thereby sealing the device (1). In some contemplated embodiments, the device (1) is sealed by engagement of the cap and collection tube using one or more of a snap closure, bayonet closure, or friction seal, or by any other closure mechanism by which a cap securely closes a vessel to seal contents inside the vessel. In preferred embodiments, mated threads on the cap (5) and collection tube (3) provide for securing the sampler wand (2) with a half-turn screw fit to the collection tube (3). As illustrated, e.g., in FIGS. 4, and 12A, in some embodiments, device (1) comprises a gasket (25) to seal the junction between cap (5) and collection tube (3).

In some embodiments, device (1) is configured to allow a user to separate sampler head (8) from cap (5), e.g., after a sampler wand (2) containing a collected sample is engaged with collection tube (3) to seal the device. For example, in some embodiments, sample wand (2) is configured to have disengagement feature, such that manipulation of sample wand (2) or of the sealed device (1) detaches a detachable sampler head (8) from the sampler wand (2). For example, in some embodiments, stem (4) comprises a break point or detachment point wherein separation occurs, e.g., when cap (5) is loosened from or further rotated with respect to collection tube (3). In some embodiments, all or part of stem (4) remains attached to detached sampler head (8), while in some embodiments, all or part of stem (4) detaches from detached sampler head (8). In some embodiments, an engagement feature of a sampler head (8) is configured to interact with an engagement feature of collection tube (3), e.g., on the bottom wall (20) and/or on displacing member (21) of the collection tube (3), so that the sampler head (8) may be reversibly or irreversibly attached to collection tube (3), e.g., upon sealing of the device. Engagement of the sampler head (8) with the collection tube (3) may be configured to facilitate separation of the sampler head (8) from the cap (5), e.g., by facilitating breakage or disengagement of the sampler head (8) from cap (5).

In some embodiments, the device further comprises an ejector movably attached to or incorporated into sampler wand (2), e.g., in the cap (5), wherein manipulation of the ejector (e.g., pressing or sliding a button, pulling a tab, rotating a portion of the cap, etc.) detaches sampler head (8) from cap (5).

As illustrated in FIGS. 12B and 12C, in some embodiments, device (1) comprises a gasket-less seal at the junction between cap (5) and collection tube (3). For example, precision molding processes may be used so that contact between surfaces on cap (5) and collection tube (3) come into direct contact to make a sealing joint (27) between the cap and collection tube. FIG. 12B shows a detail illustration of an embodiment in which the sealed joint (27) is made by contact between a flange portion (4a) of stem (4) of sampler wand (2) and the top edge (18) of collection tube (3), and FIG. 12C shows an embodiment in which the sealed joint (27) is made by contact between a sealing lip (28) on cap (5) and a sealing surface (29) on an inner side wall (30) of collection tube (3). In preferred embodiments, sealing lip (28) is an integral part of cap (5), e.g., it is formed during the molding process to produce a part of sampler wand (2), e.g., the molding of cap (5) or stem (4).

### Collection Tube

The technology provides a device (1) comprising a collection tube (3). In some embodiments, collection tube (3) has an overall tubular or cylindrical shape. However, the collection tube may have a non-circular cross-section, e.g., oval, hexagon, square, etc. In preferred embodiments, a collection tube (3) that uses a threaded closure, e.g., as illustrated in FIG. 1, is circular in cross-section on at least the portion of the collection tube that comprises tube threads (24). In preferred embodiments, the collection tube (3) comprises a displacing member (21), which is typically a protrusion from the bottom wall (20) of the collection tube (3) into the interior void (22) of the collection tube (3). See, e.g., FIGS. 1 and 3. The displacing member (21) may be hollow or solid, may be formed of the same material as collection tube (3), and may be formed at the time the tube is formed, or may be added to a flat bottom wall (20) of a collection tube after the body of the collection tube is separately formed. In preferred embodiments, the displacing member (21) is formed as an integral part of collection tube (3), formed at the same time and from the same material as collection tube (3). For example, FIG. 4A provides a cross sectional view of device (1), showing displacing member (21) formed by an indent (26) formed in bottom wall (20) of collection tube (3), wherein the collection tube side wall (17), bottom wall (20) and displacing member (21) are formed as a continuous piece of, for example, plastic. In some embodiments, indent (26) is used as a way of engaging the collection tube (3), e.g., to engage the collection tube (with or without the other components of device (1)) with a holder, rack, conveyor, mixer, gripper, rotator, etc.

In some embodiments, displacing member (21) is essentially circular in cross section and is preferably centered on a central axis of collection tube (3). In preferred embodiments, the central axis of displacing member (21) in device (1) in a closed or sealed state, e.g., as illustrated in FIG. 2, is aligned with the centers of cap (5), stem (4), and sampler head (8) on the central axis (23) of the device (1), as illustrated in FIGS. 1 and 2.

An object of the displacing member (21) is to occupy some or all of the interior void (9) of sampler head (8) of the sampler wand (2). Thus, in preferred embodiments, displacing member (21) is dimensioned to match the interior dimensions of the sampler head (8), as shown in FIG. 4A). Further, the displacing member (21) is positioned inside the bottom of collection tube (3) such that when the sampler wand (2) is fully inserted into collection tube (3) (e.g., when a cap (5) on sampler wand (2) is screwed into place to seal collection tube (3)), displacing member (21) fills most or all of the interior void (9) of the sampler head (8). Thus, during use in collecting a metered stool sample, material within sampler head (4) is expelled from the sampler head through one or more ports (11) as the displacing member (21) displaces the material. The displacing member need not expel all of the sample material from the sampler head and the amount expelled may be determined by how much, e.g., what percentage, of the volume of the sampler head (8) interior void (9) is filled by displacing member (21) when the device is sealed. In preferred embodiments, the displacing member of the collection tube fills at least 50%, 55%, 60%, 65%, 70%, 75%, 80%, 85%, 90% 95%, 96%, 97%, 98%, 99% of the interior volume of the sampler head. In some embodiments, collection tube (3) comprises a movable displacing member (21), e.g., that may be positioned in a fully or partially withdrawn position with respect to a sampler head (8) when sampler wand (2) is fully inserted into collection tube (3), and that may be moved into position, e.g., extended into the interior void (9) of the sampler head (8), thereby displacing some or all of the material in the sampler head.

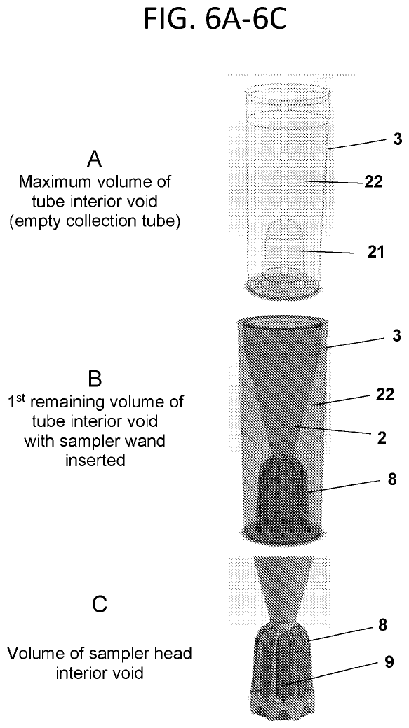

As illustrated in FIG. 6A, collection tube (3) has a maximum volume of an interior void (22) of collection tube (3) when empty. As used herein in reference to the maximum volume of the collection tube, the terms “mL” (milliliters) and “cc” (cubic centimeters) are used interchangeably, and 1 cc is equivalent to 1 mL. In preferred embodiments, the maximum volume of collection tube (3) is at least 5 mL, preferably between about 5 mL and 250 mL, including any intermediate number of mL or fractions thereof (e.g., 1 mL, 01 mL, 001 mL or multiples thereof). In preferred embodiments, a collection tube (3) has a maximum volume of between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 560, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof (e.g., 0.1 mL, 0.01 mL, 0.001 mL or multiples thereof).

As illustrated in FIG. 6B, the primary remaining volume of the interior void (22) of collection tube (3) is the maximum volume, less the volume of displacement by the stem (4) with sampler head (8). FIG. 6C illustrates the volume of interior void (9) of sampler head (8). In preferred embodiments, a secondary remaining volume of interior void (22) upon insertion of the stem and sampler head (8) is the maximum volume, less the volume of displacement by the stem (4) with sampler head (8), less the volume of the interior void (9) of sampler head (8). The secondary remaining volume is preferably at least 2 times (2×) the volume of the interior void (9) of sampler head (8), preferably at least 3×, preferably at least 4×, preferably at least 5, 6, 7, 8, 9, or 10× the volume of the interior void (9) of sampler head (8), or any intermediate fractions thereof (e.g., 4.1×, 4.2×, 4.3×, 4.4×, etc.). In other embodiments, the secondary remaining volume of the interior void (22) is the same as, or is less than, the interior void (9) of sampler head (8).

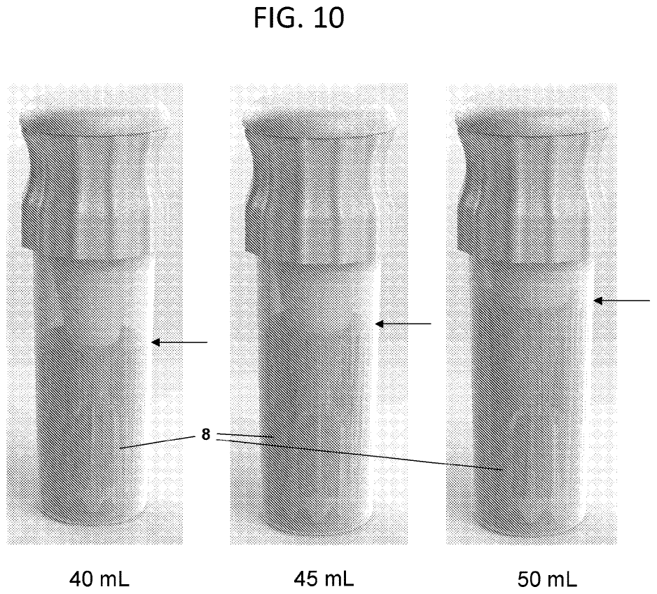

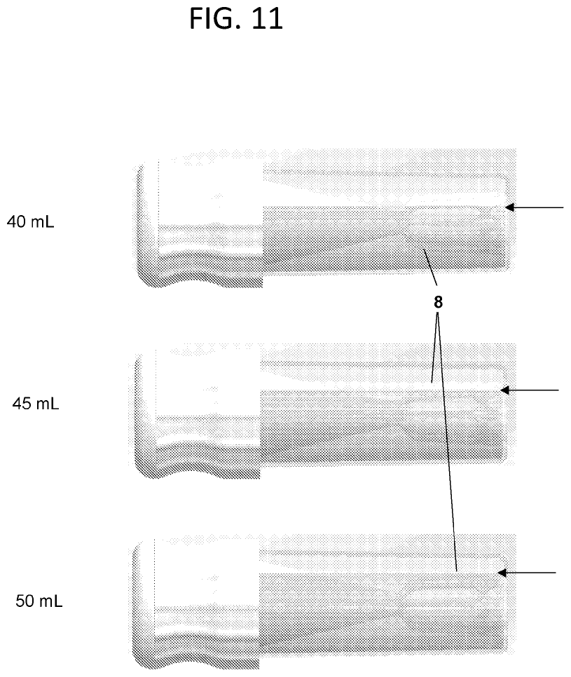

As illustrated in FIGS. 10 and 11, in preferred embodiments, the remaining interior volume is preferably large enough to hold a volume of solution, e.g., a stabilizing solution or buffer, that at least partially covers sampler head (8), preferably covering most of the sampler head (8), more preferably completely covers sampler head (8). Preferably, sampler head (8) is partially or completely covered when the sealed device containing the metered sample is horizontal, e.g., laying on its side as shown in FIG. 11. Preferably, the ratio of the volume of interior void (9) of the sampler head (8) to the volume of solution in the collection tube, whether provided as a pre-filled solution or as a solution reconstituted from dried reagents, is at least about 1:2 (v:v), preferably between 1:2 and 1:300 (v:v), preferably between 1:2 and 1:100 (v:v), preferably between 1:2 and 1:75, 1:50, 1:40, 1:30, 1:20, 1:10, or 1:5 (v:v).

In preferred embodiments, during use, some or all of the material in sampler head (8) is expelled into a surrounding fluid, e.g., a stabilizing buffer, in collection tube (3), when the sampler wand (2) is inserted into collection tube (3), e.g., when cap (5) engages to collection tube (3) to form a sealed device.

Filling of the sampler head (8) with a material such as stool, e.g., using a twisting, coring, plunging, or scraping action provides a sample having a volume or mass that is largely determined by the volume of the interior void of the sampler head (8). Expulsion of the metered sample through multiple ports (11) breaks up the metered sample and disperses it into the buffer, enhancing the stabilizing effect of the buffer.

### Materials

Some embodiments find advantages in constructing the device from various types of materials, e.g., disposable materials, recyclable materials, reusable materials, sterilizable materials, autoclavable materials, chemically inert materials, biodegradable materials, etc. The members of device (1) may be made from any material that provides structural soundness to the device as used for its intended purpose, and that is chemically and biochemically compatible with metered samples and solutions, e.g., stabilizing buffers, which may contact the device.

Members of the device may all be composed of the same material, or different members may be composed of different materials. For example, in preferred embodiments, members of device (1) that are exposed to the metered sample are typically selected to minimize or prevent binding of analytes (e.g., nucleic acids, proteins, cells) to the surfaces of these members during use, which may result in loss of the bound material. Materials are also typically selected to minimize or prevent alteration of the sample components, e.g., degradation of molecules, alteration in viability or stability of human cells, bacteria, or viruses, etc. For example, materials may be selected to avoid altering the composition of the sample components, by releasing, leaching, and/or transferring materials, such as plasticizers, coatings, or byproducts of manufacture (e.g., bisphenol A (BPA), phthalates, per- and polyfluoroalkyl substances (PFAS), fine particulates such as talcum powder, surfactants, dyes, etc.), to the sample.

In some embodiments, one or more elements of the device are made from plastics such as, e.g., polypropylene, polyethylene, polystyrene, and polytetrafluoroethylene. In some embodiments, the collection tube (3) is made from a transparent plastic, e.g., polypropylene (PP) or polyethylene (PE), while in some embodiments, a collection tube may comprise or be composed of a material that is not transparent, e.g., to limit light exposure to a contained sample. Different materials may be suitable for different applications and sample types. Embodiments of the device may comprise, for example, one or more of silicone, glass, ceramic and/or crystalline materials (e.g., alumina, hydroxyapatite, zirconia, diamond, graphite), metal materials (e.g., platinum, gold, cobalt-chromium alloy, stainless steel, titanium & titanium alloys, etc.), and/or polymers (e.g., acetal (ACL), cyclic olefin copolymer (COC), ethylene chlorotrifluoroethylene (ECTFE), ethylene propylene diene monomer rubber (EPDM) ethylene propylene rubber (EPR), ethylene tetrafluoroethylene (ETFE), fluorinated ethylene propylene (FEP), fluorinated high-density polyethylene (FLPE), high-density polyethylene (HDPE), high impact polystyrene (HIPS), low density polyethylene (LDPE), polycarbonate (PC), pothetherimide (PEI), polyethylene terephthalate (PET), polyethylene terephthalate copolymer (PETG), polymethylmethacrylate (PMMA), polymethylpentene (PMP), polypropylene (PP), polypropylene copolymer (PPCO), modified polyphenylene ether (PPE), polystyrene (PS), polysulfone (PSF), polyvinylidenedifluoride (PVDF), tetrafluoroethylene (PTFE/TFE), thermoplastic elastomer (TPE), trimethylcarbonate (TMC), TMC NAD-lactide, etc.). In some embodiments, components of sampler wand (2) and collection tube (3) are made from the same material. For example, in certain embodiments, the same material, e.g., polypropylene polyethylene, or one of the polymers discussed above, is used for multiple parts but one or more parts are differently colored or have a different level of transparency. For example, in some embodiments, a collection tube (3) is made of clear polypropylene and parts of sampler wand (2) (e.g., the cap (5), stem (4) and/or sampler head (8)) are composed of opaque polypropylene, e.g., white polypropylene.

In some embodiments, components of sampler wand (2) and collection tube (3) are made from different materials. For example, in some embodiments, a collection tube (3) is made of clear polypropylene and parts of sampler wand (2) (e.g., the cap (5), stem (4) and/or sampler head (8)) are composed of opaque polyethylene, e.g., white polyethylene, while in some embodiments, a collection tube (3) is made of opaque material, e.g., white, amber, brown, or black polyethylene or polypropylene, e.g., to shield contents of the collection tube (3) from view or from light, and parts of sampler wand (2) (e.g., the cap (5), stem (4) and/or sampler head (8)) are composed of clear material, e.g., clear polypropylene, polymethylpentene, or polycarbonate

In some embodiments, sampler wand (2) comprises a mixture of different materials. For example, in some embodiments, the cap (5) is made of one material, and stem (4) and/or sampler head (8) are made from one or more different materials.

The materials that find use in the device (1) are not limited to any particular selection or combination of suitable materials used for the parts of the device. In some embodiments, the materials and compositions are chosen to provide a shelf-life for the device of at least 2 years. In some embodiments, materials for the device are selected to block light and/or ultraviolet radiation. For example, amber or opaque materials may be selected. In some embodiments, materials may be selected to allow inspection of samples contained therein, e.g., optically, or under different lighting conditions. Thus, in some embodiments, materials may be selected for their optical properties, e.g., degree of transparency under different lighting conditions, e.g., in light withing the daylight spectrum, and/or under UV lighting. For example, in some embodiments, materials may be selected to fluoresce or to lack fluorescence under UV light.

As illustrated, e.g., in FIGS. 4, and 12A, in some embodiments, device (1) comprises a gasket (25) to seal the junction between cap (5) and collection tube (3). Any suitable material, typically a polymeric material may be used. In some embodiments, the gasket comprises low density polyethylene (LDPE), silicone, nitrile, EPDM (ethylene propylene diene monomer) rubber, vinylidene fluoride diene rubber (VITON), etc. In certain preferred embodiments, the gasket comprises multiple plies. For example, in some embodiments, a gasket is a three-ply co-extruded material consisting of a foamed LDPE core sandwiched between two layers of solid LDPE, e.g., TRISEAL F-217® (Tri-Seal, Blauvelt, NY).

In some embodiments, the sampler head (8) material may be selected to provide a particular degree of friction with respect to a sample, e.g., to retain sample material such as stool during the sampling process. In some embodiments, the interior surface of the sampler head (8) provides friction to retain the metered sample, e.g., during removal of the metered sample from the stool specimen. For example, in some embodiments, the number and arrangement of ports (11) are selected to enhance retention of a very soft sample, e.g., a semiliquid sample, within the interior sampler head (8).

In some embodiments, one or more materials or surface on materials in device (1) may be selected to provide a smooth surface, e.g., to enhance release of sample material from sampler head (8), collection tube (3), and/or stem (4) may have a smooth and/or treated surface to enhance release of material. In some embodiments, one or more of the device surfaces comprise a non-stick or hydrophobic layer or coating.

In some embodiments, materials of cap (5) may be selected for enhanced gripping. For example, in some embodiments, members of cap (5) may comprise a thermoplastic vulcanizate (TPV), e.g., Santoprene™ TPV (Celanese Corp.) to have a rubber-like grip and feel. Santoprene™ TPV is fully dynamically vulcanized EPDM (ethylene propylene diene monomer) rubber in a thermoplastic matrix of polypropylene (PP). In preferred embodiments, raised features (16) of cap (5), e.g., as shown in FIG. 2, are fully or partially formed from a TPV.

In some embodiments, cap (5) and/or collection tube (3) are configured to facilitate automated processing, e.g., opening, closing, and/or transferring the device by mechanical or robotic means. For example, in some embodiments, cap (5) comprises one or more features configured to engage with a cap driver device, e.g., raised ridges or grooves on the sides of cap (5) and/or collection tube (3); raised element(s) and/or indented feature(s) (e.g., ridge, blade, cross, hexagon, square, or star-shaped elements, etc.) on the top of cap (5), e.g., for automatically or robotically rotating cap (5) with respect to the collection tube (3) using, e.g., a cap driver.

### Solutions

In some embodiments, the device comprises a solution in the collection tube (3), and in some embodiments, the device comprises a form of reagent, e.g., one or more reagents dried, e.g., as a powder, cake, or coating, or as an encapsulated composition that can be dissolved, suspended, or otherwise disrupted with a fluid to provide a solution in the collection tube (3). In some embodiments, the solution (provided as a solution or as a dried form to form a solution) is a stabilizing buffer providing a buffer and one or more salts, e.g., sodium chloride. In certain embodiments, the stabilizing solution comprises Tris buffer and EDTA, e.g., 500 mM Tris, pH 9.0, 150 mM EDTA, and 10 mM NaCl. In some embodiments, other salts may be used, e.g., Hanks Balanced Salt Solution (HBBS).

### HBSS:

| | | |
|---|---|---|
| CaCl₂ dihydrate | 0.185 | g/L |
| MgSO₄ (anhydrous) | 0.09767 | g/L |
| KCl | 0.4 | g/L |
| KH₂PO₄ (anhydrous) | 0.06 | g/L |
| NaHCO₃ | 0.35 | g/L |
| NaCl | 8.0 | g/L |
| Na₂PO₄ (anyhydrous) | 0.04788 | g/L |
| D-Glucose | 1.0 | g/L |

pH 7.4 ± 0.2

In some embodiments, a stabilizing solution may comprise reagents selected from a protoporphyrin; a polyvalent cation; a sugar or polysaccharide and, optionally, a polyvalent cation; an osmolyte; and a horse radish peroxidase (HRP) stabilization component and, optionally, a polyvalent cation, e.g., as described in WO 2019/190787, which is incorporated herein by reference for all purposes. For example, one such exemplary solution may comprise a buffer and a protoporphyrin complexed with Cr³⁺ or Co³⁻, wherein the concentration of the protoporphyrin complexed with Cr³⁺ is 1.25-5 μM and the concentration of the protoporphyrin complexed with Co³⁺ is 2.5-10 μM.

In some embodiments, a stabilizing solution may comprise one or more of a surfactant (e.g., Tween-20) and a nuclease inhibitor (e.g., RNase inhibitor, proteinase K, a chelator). In some embodiments, the solution comprises one or more of sarkosyl, and a chaotropic salt (e.g., guanidine thiocyanate).

The solution comprises components to break up, solubilize, and/or suspend the metered sample such that withdrawal of a portion of the mixture provides an aliquot suitable for analysis. In addition, the solution comprises components to stabilize, preserve, and/or protect the resulting suspension so that the analytes to be tested (e.g., cells, RNA, DNA, proteins) do not degrade or become damaged between the time the sample is acquired and the time the sample is tested. The solution thus helps to ensure that the analysis of the metered sample accurately reflects the analytes (e.g., RNA, DNA, proteins) present in the sample when the sample was acquired. The solution or the dried agents may also comprise components to neutralize or bind to substances that may inhibit downstream assays, e.g., polyvinylpyrrolidone and/or polyvinylpolypyrrolidone which bind to phenolic substances. In some embodiments, one or more of the reagents, e.g., polyvinylpolypyrrolidone, are provided in a form that is not soluble in the solution. In some embodiments, preferably in embodiments wherein the device contains dried reagents, the device contains a desiccant. The technology contemplates any solution useful to preserve or prepare the metered sample, e.g., for a particular type of analysis.

### Use of the Device

The device is designed to provide an easy way to collect and produce fecal samples for analysis. Accordingly, it is designed for use by both the sample provider and the sample tester. The sample provider may be, in some uses of the device, a person who does not have medical or clinical training, and thus the device is easy to use for such lay users in acquiring the sample. The device is therefore particularly suitable for use remotely, e.g., in the home or residential facility, or anywhere outside a medical clinic or laboratory, or for use in the field, e.g., for collecting environmental or industrial samples. Thus, in some embodiments the collection tube (3) is composed of opaque material, or is covered, e.g., with a label, such that the interior components (e.g., the stem and sampler head of the sampler wand) are substantially blocked from view of a user, e.g., to avoid confusion regarding the proper use of the device.

At the same time, the device is designed within the appropriate tolerances and with a design that allows for precise and accurate analysis of the sample by a human or machine tester. Generally, the device will be provided to the sample provider in an assembled form—e.g., the collection tube (3) is pre-filled with a volume of solution, and the cap (5) of the sampler wand (2) is secured to the collection tube such that the device (1) is sealed.

Upon producing a stool, the sample provider removes the sampler wand (2) from the collection tube (3) and places the distal edge (12) of the sampler head (8) at a site on the stool specimen. Using a twisting action, the sample provider cuts the distal edge (12) of the sampler head (8) into the specimen, preferably to capture a roughly cylindrical portion of the specimen within the interior void (9) of the sampler head (8), in a “coring” fashion. Preferably, the sample provider collects sufficient stool material to fill the interior void (9) of sampler head (8), either at a single locus on the specimen, or by successive coring at multiple different loci on the specimen. In some embodiments, teeth (13) on the sampler head (8) may also be used to loosen sample material and/or to collect sample material by scraping, e.g., scraping across a specimen to obtain a sample from multiple locations. In preferred embodiments, removing the sampler head from the specimen with a twisting action results in release of the sampler head (8) from the specimen with a minimum of excess stool material on the exterior of the sampler head (8).

The sampler wand (2) is then re-inserted into the collection tube (3), cap threads (7) are engaged with the tube threads (24) and the cap (5) is rotated with respect to the collection tube (3) until it can no longer be turned, such that the device (1) is completely sealed, and fluid cannot escape.

As the cap (5) is replaced on and secured to the collection tube (3), displacing member (21) is aligned with the sampler head (8) and is forced into sampler head (8) through sampler head opening (14). The displacing member (21) then presses stool material out of the sampler head (8) through ports (11), into a solution, e.g., a buffer contained in the collection tube (3). Forcing sample material through the ports serves to break up and reduce the particle size of the sample, and the solution then acts to further break up, solubilize, and/or suspend the metered sample. In addition, the solution typically comprises components to stabilize, preserve, and/or protect the suspension. In some embodiments, the sample provider further mixes the solution and the metered sample, e.g., by shaking, rotating, or otherwise agitating the sealed device. The sample provider then returns the sealed device (1) containing the metered sample to a laboratory, clinic, or other location for analysis. In some embodiments, the device is designed to be appropriate for return by mail or shipping service.

Next, the testing facility retrieves a portion of the stool suspension for analysis. In some embodiments, the sealed container is shaken, rotated, or vortexed to ensure that the metered sample is completely dispersed or homogenized in the solution. A user or machine withdraws a portion of the suspension (e.g., by a syringe needle or a pipette tip) to collect an aliquot of the suspension for further processing and testing. As illustrated in FIG. 9, the sampler wand (2) is removed from the collection tube (3) to access the mixture of buffer and stool in the collection tube (3). As shown, embodiments of the collection tube (3) are configured to allow a pipet tip to reach the bottom of the collection tube unimpeded, facilitating withdrawal of an aliquot of or essentially all of the suspended material. In some embodiments, a suspension is removed from collection tube (3) without removal of sampler wand (2). For example, in some embodiments, cap (5) or collection tube (3) comprises an openable port that provides access to fluid or suspension contained in the device. In some embodiments, a device may be punctured, e.g., with a needle or canula, for access to fluid or suspension contained in the device. In some embodiments, device (1) comprises a penetrable seal through which a portion of the suspension is obtained. Some embodiments provide that the seal is made from foil. However, other materials are appropriate for the device provided the material seals the sample collection chamber while also being penetrable (e.g., by a pipette tip or by a syringe needle) such that a human or machine obtains a portion of the contents sealed within device (1). Some non-limiting examples of materials provided in various embodiments include foil paper, rubber, wax, and plastic, and combinations thereof, e.g., in layered or laminated forms.

In some embodiments, a cap having an attached sampler wand is replaced by a different cap, e.g., after expulsion of the sample from the sampler head into a solution in the collection tube. Such a replacement cap is preferably one that engages with the collection tube (3) to form a sealed container, but that does not have a sampler wand attached, thereby facilitating or simplifying additional processing steps, e.g., centrifugation and supernatant or pelleted sample acquisition, using the original collection tube (3).

In some embodiments, some or all of the suspension is poured from the collection tube (3) to a different container or device for further processing steps. After the required aliquot of the suspension is withdrawn, the device may be resealed and any remaining stool sample suspension may then be stored (e.g., heated, ambient, refrigerated, or frozen storage) or used in another process, or discarded, as appropriate.

While use of the device for stool sample collection is described as one embodiment, the device (1) finds application in sampling any type of similar soft materials, e.g., foods, environmental samples (e.g., soil or biological materials), waste or industrial materials, etc.

### Systems and Kits

The technology finds use in kits comprising embodiments of the devices described and, in some embodiments, optional components such as, e.g., an instruction for use (e.g., providing the steps of a related method) and related packaging for storage, shipping, and the like. Embodiments of the kits may comprise one or more solutions, e.g., comprising a stabilizing reagent, a buffer, a salt, or a preservative for using with (e.g., for treating, homogenizing, preserving, or storing) the collected metered sample and the analytes it contains.

Kits may also comprise other components useful for catching a stool specimen, such as a collection system for use with a commode or toilet for conveniently securing a stool specimen produced by a subject during defecation, such that the metered sample may be collected from the specimen using the technology. Many different suitable commode specimen collectors, e.g., paper or plastic bowls, slings, webs, bags, or other receptacles that fit to a commode or toilet, are available through medical suppliers, pharmacies, and scientific supply companies, or directly from manufacturers, e.g., Therepak Corp., Zymo Research; Alpha Labs; Excretas, HyStool; Abexx; Ability Building Community; or Takahashi Keisei Corp. The stool specimen container of U.S. Pat. No. 10,265,054 is also suitable for use.

Kits may also provide other items to facilitate use of device, or shipping and handling of the device before or after use. For example kits may provide a holder or rack, to hold the device during use (e.g., to stably hold the collection tube in a vertical position, to avoid spillage when the device is not sealed) and/or to secure the sealed device in a shipping container.

Aspects of the technology are illustrated in U.S. Provisional Application Ser. No. 63/381,031, filed Oct. 26, 2022, U.S. Design Application Ser. No.______, 29/867,482 filed Oct. 26, 2022, and Industrial Design Application Ser. No. WIPO132201/970181898, filed Apr. 26, 2023, now International Registration No. DM/231747, each of which is incorporated herein by reference in its entirety, for all purposes.

### EXPERIMENTAL

As discussed above, technology herein is directed to overcoming the challenge of providing a sample collection device that can be reliably used by any subject in a diverse population of subjects to collect a metered sample of material, e.g., stool, in a stabilized manner that is suitable for transport, e.g., from a subject's home to a medical laboratory using standard commercial shipping methods or public mail. Exemplary embodiments of the device of the technology are shown in FIGS. 1-12.

### Example 1

### Collection of Metered Samples

An embodiment of the sample collection device as depicted in FIGS. 3A and 3B was used to collect stool material from surfaces and interiors of 20 different stool specimens having different consistencies. The mass of each metered sample collected from each stool specimen was measured to determine the range of sample sizes produced using the device, with the results shown in the table below:

| | Mass |
|---|---|
| Specimen | collected (g) |
| 1 | 10.44 |
| 2 | 11.40 |
| 3 | 12.15 |
| 4 | 11.69 |
| 5 | 8.61 |
| 6 | 12.83 |
| 7 | 8.66 |
| 8 | 9.57 |
| 9 | 12.79 |
| 10 | 11.16 |
| 11 | 10.39 |
| 12 | 10.09 |
| 13 | 10.87 |
| 14 | 10.83 |
| 15 | 10.00 |
| 16 | 10.52 |
| 17 | 11.08 |
| 18 | 13.81 |
| 19 | 10.53 |
| 20 | 8.67 |
| Average: | 10.80 |

These data show that the mass of stool collected from the different specimens averaged 10.8 grams per specimen, which is within a suitable range for use in stool sample processing and testing methods (e.g., methods such as those described by DA Ahlquist, et al., Gastroenterology 2012; 142:248-256, which is incorporated herein by reference in its entirety, for all purposes.

### Example 2

Nucleic Acid Capture and Testing from Collected Metered Samples

An embodiment of the sample collection device as depicted in FIGS. 3A and 3B was used to collect stool material from 10 stool specimens (5 samples from subjects with a condition associated with particular target nucleic acids in stool (“cases”) and 5 control samples). Sampler wands comprising the collected samples were inserted into collection tubes containing 40 mL of buffer. The mixtures of collected metered sample and buffer were processed and nucleic acids were isolated from stool supernatants by direct capture of target sequences by hybridization with oligonucleotide probes, as described in DA Ahlquist, et al., supra. Three human target nucleic acids (A, B, and C) and one human reference nucleic acid (R) were isolated from each sample. The nucleic acids were assayed using a PCR-flap assay, as described, e.g., in WO 2021/041726 to S. Morris, et al., which is incorporated herein by reference in its entirety for all purposes. Elevated amounts of at least one of nucleic acids A, B, or C were taken as being positively indicative of the condition in the subject (indicated as “POS” in the Assay Call column). The assay results are summarized in the table below:

| | Mean | Mean | Mean | Mean | |
|---|---|---|---|---|---|
| | Nucl. | Nucl. | Nucl. | Nucl. | |
| | Ac. A | Ac. B | Ac. C | Ac. R | Assay |
| Sample | (Strands) | (Strands) | (Strands) | (Strands) | Call |
| CASE | 960 | 9724 | 9618 | 22311 | POS |
| CASE | 11 | 20 | 20 | 1200 | POS |
| CASE | 4 | 500 | 610 | 3366 | POS |
| CASE | 36 | 23 | 258 | 7365 | POS |
| CASE | 11 | 28 | 52 | 1110 | POS |
| Contol | 12 | 16 | 15 | 6147 | NEG |
| Contol | 0 | 1 | 0 | 1275 | NEG |
| Contol | 4 | 3 | 9 | 3222 | NEG |
| Contol | 0 | 8 | 0 | 10746 | NEG |
| Contol | 0 | 4 | 8 | 8939 | NEG |

Each metered sample yielded enough stool supernatant to assay the panel of 4 nucleic acids listed above at least twice. These data demonstrate that sufficient quantities of stool were obtained using the sample collection device of the technology to quantitatively assay specific human target nucleic acids with sufficient sensitivity and specificity for diagnostic applications.

All literature and similar materials cited in this application, including but not limited to, patents, patent applications, articles, books, treatises, manufacturer publications and product literature, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

What is claimed is:

1. A device for collecting and containing a metered sample, the device comprising a sampler wand comprising a sampler head, wherein the sampler head comprises:
i) a sampler head side wall comprising a plurality of ports therethrough;
ii) a sampler head closed end; and
iii) a sampler head opening bounded by a distal edge;
wherein the sampler head side wall, sampler head closed end and sampler head opening define and contain a sampler head interior void having an interior volume.

2. The device of claim 1, wherein the sampler head opening is on a plane defined by the distal edge.

3. The device of claim 1, wherein the sampler head has a sampler head central axis, wherein the sampler head opening has a center point on the sampler head central axis.

4. The device of any one of claims 1-3, wherein the sampler wand comprises a cap.

5. The device of claim 4, wherein the cap is attached to the sampler head, preferably to the sampler head closed end, by a stem.

6. The device of claim 4 or claim 5, wherein the cap has a center point on a cap central axis, and wherein the sampler wand has a wand central axis defined by the center point of the cap and the center point of the sampler head opening.

7. The device of any one of claims 1-6, wherein the distal edge of the sampler head comprises one or more teeth.

8. The device of any one of claims 1-7, wherein the sampler head interior void is sized to contain from about 0.1 grams to about 50 grams of sample material, preferably stool sample material.

9. The device of claim 8, wherein the sampler head interior void is sized to contain about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams, including any intermediate fractions thereof, of sample material, preferably stool sample material.

10. The device of claim 9, wherein the sampler head interior void is sized to contain about 1, 2, 3, 4, 5, 6, 7, 8, 9, 10, 11, 12, 13, 14, or 15 grams, including any intermediate fractions thereof, of sample material, preferably stool sample material.

11. The device of any one of claims 4-10, further comprising a collection tube comprising a bottom wall joined to a tube wall, wherein a top edge of the collection tube defines a tube opening.

12. The device of claim 11, wherein the cap comprises a cap engagement portion and the collection tube comprises a mated engagement portion.

13. The device of claim 12, wherein the cap engagement portion comprises cap threads and wherein the mated engagement portion comprises tube threads.

14. The device of claim 12 or claim 13, wherein when the cap engagement portion and the mated engagement portion are engaged, the sampler wand and the collection tube form a sealed device.

15. The device of claim 14, wherein the collection tube has an interior void bounded by the tube wall, the bottom wall, and a plane defined by the top edge, the tube interior void having an interior void maximum volume of at least 5 mL, preferably between about 5 mL and 250 mL, including any intermediate number of mL or fractions thereof, preferably between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof.

16. The device of claim 14 or claim 15, wherein the bottom wall of the collection tube comprises a displacing member projecting into the interior void of the collection tube.

17. The device of claim 16, wherein the collection tube has a tube central axis defined by center points of the collection tube opening and of the displacing member, and wherein the cap has a center point on a cap central axis, and wherein the sampler wand has a wand central axis defined by the center point of the cap and the center point of the sampler head opening.

18. The device of claim 17, wherein the cap has a center point on a cap central axis, and wherein the sampler wand has a wand central axis defined by the center point of the cap and the center point of the sampler head opening, and wherein, when the sampler wand and the collection tube form a sealed device, the wand central axis and the tube central axis are collinear.

19. The device of any one of claims 16-18, wherein, when the sampler wand and the collection tube form a sealed device, the displacing member of the collection tube is positioned within the sampler head and fills at least a portion of the sampler head interior void.

20. The device of claim 19, wherein, when the sampler wand and the collection tube form a sealed device, the displacing member of the collection tube fills at least 50%, 55%, 60%, 65%, 70%, 75%, 80%, 85%, 90% 95%, 96%, 97%, 98%, 99% of the interior volume of the sampler head interior void.

21. The device of claim 20, wherein the displacing member of the collection tube fills substantially all of the sampler head interior void.

22. The device of any one of claims 16-21, wherein the device has a primary remaining volume of the interior void of the collection tube that equals the interior void maximum volume less the interior void volume displaced by a sampler head and sampler stem.

23. The device of claim 22, wherein a ratio (v:v) of the interior void of the sampler head to the primary remaining volume of the interior void of the collection tube is at least about 1:2, preferably between about 1:2 and 1:300, preferably between about 1:2 and 1:100, preferably between about 1:2 and 1:75, 1:50, 1:40, 1:30, 1:20, 1:10, or 1:5.

24. The device of any one of claims 16-23, wherein the device further comprises a volume of solution contained in the collection tube.

25. The device of claim 24, wherein a ratio (v:v) of the volume of the interior void of the sampler head to the volume of solution in the collection tube is at least about 1:2, preferably between 1:2 and 1:300, preferably between 1:2 and 1:100, preferably between 1:2 and 1:75, 1:50, 1:40, 1:30, 1:20, 1:10, or 1:5.

26. The device of claim 24 or claim 25, wherein the solution comprises one or more agents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

27. The device of any one of claims 16-23, wherein the device further comprises at least one dried agent contained in the collection tube.

28. The device of claim 27, wherein the at least one dried agent comprises one or more reagents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt;
XV) an inhibitor binding agent; and
xvi) a desiccant.

29. The device of any one of claims 22-28, wherein the device has a secondary remaining volume of the interior void of the collection tube that equals the maximum volume of the interior void of the collection tube less the volume displaced by a sampler head and sampler stem, less the volume of the sampler head interior void.

30. The device of claim 29, wherein a ratio (v:v) of the interior void of the sampler head to the secondary remaining volume of the interior void of the collection tube is at least about 1:2, preferably between about 1:2 and 1:300, preferably between about 1:2 and 1:100, preferably between about 1:2 and 1:75, 1:50, 1:40, 1:30, 1:20, 1:10, or 1:5.

31. A method of collecting a metered sample of material, comprising:
a) providing a device comprises a sampler wand comprising a sampler head, wherein the sampler head comprises:
i) a sampler head side wall comprising a plurality of ports therethrough;
ii) a sampler head closed end; and
iii) a sampler head opening bounded by a distal edge;
wherein the sampler head side wall, sampler head closed end and sampler head opening define and contain a sampler head interior void having an interior volume; and
b) collecting an amount of the material within the interior void of the sampler head.

32. The method of claim 31, wherein the sampler head opening is on a plane defined by the distal edge.

33. The method of claim 31 or claim 32, wherein the sampler wand comprises a cap.

34. The method of claim 33, wherein the cap is attached to the sampler head, preferably to the sampler head closed end, by a stem.

35. The method of claim 33 or claim 34, wherein the cap has a center point on a cap central axis, and wherein the sampler wand has a wand central axis defined by the center point of the cap and the center point of the sampler head opening.

36. The method of any one of claims 31-35, wherein the distal edge of the sampler head comprises one or more teeth.

37. The method of any one of claims 31-36, wherein the sampler head interior void is sized to contain from about 0.1 grams to about 50 grams of sample material, preferably stool sample material.

38. The method of claim 37, wherein the sampler head interior void is sized to contain about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams, including any intermediate fractions thereof, of sample material, preferably stool sample material.

39. The method of claim 37, wherein the sampler head interior void is sized to contain about 1, 2, 3, 4, 5, 6, 7, 8, 9, 10, 11, 12, 13, 14, or 15 grams, including any intermediate fractions thereof, of sample material, preferably stool sample material.

40. The method of any one of claims 33-39, wherein the device further comprises a collection tube comprising a bottom wall joined to a tube wall, wherein a top edge of the collection tube defines a tube opening.

41. The method of claim 40, wherein the cap comprises a cap engagement portion and the collection tube comprises a mated engagement portion.

42. The method of claim 41, wherein the cap engagement portion comprises cap threads and wherein the mated engagement portion comprises tube threads.

43. The method of claim 41 or claim 42, wherein, when the cap engagement portion and the mated engagement portion are engaged, the sampler wand and the collection tube form a sealed device.

44. The method of any one of claims 40-43, further comprising:
c) inserting the sampler head into the collection tube.

45. The method of claim 44, wherein the collection tube has an interior void bounded by the tube wall, the bottom wall, and a plane defined by the top edge, the tube interior void having a maximum volume of at least 5 mL, preferably between about 5 mL and 250 mL, including any intermediate number of mL or fractions thereof, preferably between about 10 mL and 100 mL, including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 99.999 mL, including any intermediate fractions thereof.

46. The method of any one of claims 40-45, wherein the bottom wall of the collection tube comprises a displacing member projecting into an interior void of the collection tube.

47. The method of claim 46, wherein the collection tube has a tube central axis defined by center points of the collection tube opening and of the displacing member.

48. The method of claim 47, wherein the cap has a center point on a cap central axis, and wherein the sampler wand has a wand central axis defined by the center point of the cap and the center point of the sampler head opening, and wherein, when the sampler wand and the collection tube form a sealed device, the wand central axis and the tube central axis are collinear.

49. The method of any one of claims 46-48, wherein, when the sampler wand and the collection tube form a sealed device, the displacing member of the collection tube is positioned within the sampler head and fills at least a portion of the sampler head interior void, whereby at least a portion of the amount of material collected in the sampler head interior void is expelled from the sampler head interior void through a port in the sampler head and into the collection tube, wherein the metered sample of the material is selected from:
the amount of material contained in the sampler head interior void; and
the amount of material expelled from the sampler head interior void.

50. The method of claim 49, wherein the device further comprises a volume of solution contained in the collection tube.

51. The method of claim 50, wherein a ratio of the volume of the metered sample to the volume of solution in the collection tube (v:v), or a ratio of a mass of metered sample to the volume of solution in the collection tube (w:v) is at least about 1:2, preferably between 1:2 and 1:300, preferably between 1:2 and 1:100, preferably between 1:2 and 1:75, 1:50, 1:40, 1:30, 1:20, 1:10, or 1:5.

52. The method of claim 50 or claim 51, wherein the solution comprises one or more reagents selected from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

53. A kit for collecting a metered sample of material, comprising:
i) a device of any one of claims 1-30; and
ii) an agent selected from:
a solution, and
a dried agent.

54. The kit of claim 53, wherein the device is a sealed device containing the agent.

55. The kit of claim 53 or claim 54, wherein the agent is a solution comprising one or more reagents from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt; and
XV) an inhibitor binding agent.

56. The kit of claim 53 or claim 54, wherein the agent is a dried agent comprising one or more reagents from the group consisting of:
i) a buffer
ii) a salt;
iii) a preservative;
iv) a detergent;
v) a sugar or polysaccharide;
vi) a protoporphyrin;
vii) a polyvalent cation;
viii) an osmolyte;
ix) a horse radish peroxidase (HRP) stabilization component;
x) a surfactant;
xi) a nuclease inhibitor;
xii) a protease inhibitor
xiii) a chelator;
xiv) a chaotropic salt;
XV) an inhibitor binding agent; and
xvi) a desiccant.

57. The kit of any one of claims 53-56, further comprising one or more of:
a) packaging for shipping, storage, or mailing the device;
b) printed instructions for collecting a metered sample using the device;
c) a collection system for holding specimen material while collecting a metered sample;
d) a holder for holding an unsealed device during collection of a metered sample from a specimen; and
e) a holder for securing a sealed device in a shipping container.

58. A method of processing a stool sample, the method comprising,
a) obtaining a stool sample by the method of any one of claims 31-52;
b) testing the stool sample for an amount and/or presence of one or more analytes, wherein the analytes comprise one or more of the following:
i) a nucleic acid
ii) a protein
iii) a lipid
iv) a carbohydrate, and
v) a metabolite.
